(19) United States
(12) Patent Application Publication
(10) Pub. No.: US 2009/0258853 A1
(43) Pub. Date: Oct. 15, 2009

(54) Somatostatin Agonists

(76) Inventors: Brian Eastman, San Diego, CA (US); Craig W. Lindsley, Brentwood, TN (US); Scott E. Wolkenberg, Jenkintown, PA (US); Zhijian Zhao, Wilmington, DE (US)

Correspondence Address: MERCK AND CO., INC, P O BOX 2000, RAHWAY, NJ, 07065-0907, US

(21) Appl. No.: 12/225,027

(22) PCT Filed: Mar. 9, 2007
(86) PCT No.: PCT/US2007/006112

§ 371 Date: Sep. 10, 2008

Related U.S. Application Data
(60) Provisional application No. 60/781,787, filed on Mar. 13, 2006.

(51) Int. Cl. A61K 31/4709; C07D 401/14; A61K 31/506

(52) U.S. Cl. 514/210.21; 546/153; 514/312; 514/274; 544/316

(57) ABSTRACT

This invention relates to compounds which are agonists of somatostatin and selective toward somatostatin receptor subtype SSTR2. The compounds are useful in the treatment and prevention of diabetes, and diabetes-related pathologies, including retinopathy, neuropathy and nephropathy. Many of the compounds are orally active. Thus, it is an object of this invention to describe such compounds. It is a further object to describe the specific preferred stereoisomers of the somatostatin agonists. A still further object is to describe processes for the preparation of such compounds. Another object is to describe methods and compositions which use the compounds as the active ingredient thereof.

This application claims the priority of patent application 60/781,787 filed on Mar. 13, 2006.

### BACKGROUND OF THE INVENTION

Somatostatin (SST) is a widely distributed peptide occurring in two forms SST-14 (with 14 amino acids) and SST-28 (with 28 amino acids). SST has multiple functions including modulation of secretion of growth hormone, insulin, glucagon, and gastric acid, in addition to having potent anti-proliferative effects.

The mechanism of action of somatostatin is mediated via high affinity membrane associated receptors. Five somatostatin receptors (SSTR₁-5) are known (Reisine, T.; Bell, G. I. Endocrine Reviews 1995, 16, 427-442). All five receptors are heterogeneously distributed and pharmacologically distinct. The availability of these receptors now makes it possible to determine selectives among the sub-types to guide potential clinical applications. For example, studies utilizing subtype selective peptides have provided evidence that somatostatin subtype 2 receptors (SSTR₂) mediates the inhibition of growth hormone release from the anterior pituitary and glucagon release from the pancreas. Preclinical and clinical evidence suggests that growth hormone plays a causative role in diabetic complications such as diabetic retinopathy (Frystyk, J. Hormone and Metabolism Research 2005, 37, Supplement 1: 44-48). See also WO2005097142 and WO2004032940. Somatostatin's regulation of glucagon and growth hormone secretion via SSTR₂ implies the usefulness of SSTR₂ selective analogs in the treatment of diabetes and diabetes-related pathologies, including retinopathy, neuropathy and nephropathy and many of the compounds of this invention have that selectivity. In addition, somatostatin and SSTR₂ have been implicated in a variety of other biological processes such as nociception, inflammation and cell proliferation. Therefore, the novel compounds described herein may also be useful in the therapy of a variety of conditions which include acromegaly, arthritis, cancer, pain, diarrhea, inflammatory bowel disease, irritable bowel syndrome and restenosis. The compounds of this invention are remarkably reduced in size in comparison with the natural hormone and its peptide analogs such as octreotide and seglitide, which allows ease of formulation

### SUMMARY OF THE INVENTION

This invention relates to compounds which are agonists of somatostatin and selective toward somatostatin receptor subtype SSTR₂. The compounds are useful in the treatment and prevention of diabetes, and diabetes-related pathologies, including retinopathy, neuropathy and nephropathy. Many of the compounds are orally active. Thus, it is an object of this invention to describe such compounds. It is a further object to describe the specific preferred stereoisomers of the somatostatin agonists. A still further object is to describe processes for the preparation of such compounds. Another object is to describe methods and compositions which use the compounds as the active ingredient thereof. Further objects will become apparent from reading the following description.

### DETAIL DESCRIPTION OF THE INVENTION

The compounds, their pharmaceutically acceptable salts, esters, enantiomers, diastereomers or mixtures thereof of the present invention are those of the general structural Formula I:

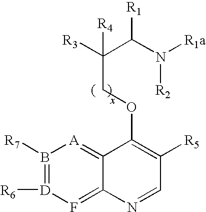

wherein:
B and D independently represent carbon and nitrogen, A and F independently represent CH and nitrogen, provided that no more than 2 of A B, D and F are nitrogen at the same time;
R₁ and R₁a independently represent hydrogen, C₁-C₁₂ alkyl, (CH₂)ₘC₃-C₈ cycloalkyl; CF₃, CF₂H, CFH₂ or
R₁ and R₁a together with the nitrogen that R₁a is attached form a monocyclic or bicyclic heterocycle with 4-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR₁S(O)ₙalkyl,
R₂ represents hydrogen, C₁-C₁₂ alkyl, (CH₂)ₘC₃-C₈ cycloalkyl, COOR₁, said alkyl optionally substituted with 1 to 3 groups of halogen, C₁₋₆ alkyl, C₁₋₃ alkoxy, hydroxyl, CN, CF₃, (CH₂)ₘN(R₁)₂, (CH₂)ₘCOOR₁, C(O)N(R₁)₂, SO₂R₁, (CH₂)ₘS(O)ₙNR₁R₂, (C(NH)N(R₁)₂);
R₁ₐ and R₂ together with the nitrogen they are attached to form a monocyclic or bicyclic heterocycle with 4-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR₁, S(O)ₙalkyl;
R₃ and R₄ independently represent hydrogen, halogen, or C₁-C₁₂ alkyl; or
R₃ and R₄ together form a monocyclic or bicyclic carbocyclic or heterocyclic ring with 4-7 members in each ring and optionally containing one to three heteroatoms selected from N, O and S, said monocylcic or bicyclic carbocycle or heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR₁, S(O)ₙalkyl; or
R₅ represents (CH₂)ₘC₆₋₁₀ aryl, (CH₂)ₘC₅₋₁₀ heterocyclyl, said aryl and heterocyclyl optionally substituted with 1 to 3 groups of halogen, C₁₋₆ alkyl, (CH₂)ₘC₃₋₇ cycloalkyl, CN, (CH₂)ₘOR₁, (CH₂)ₘCF₃, (CH₂)ₘCOOR₁₁C(O)N(R₁)₂, (CH₂)ₘS(O)ₙR₁; (CH₂)ₘS(O)ₙNR₁R₂; (CH₂)ₘ[NR₁]S(O)ₙNR₁R₂; (CH₂)ₘ[NR₁]S(O)ₙR₁;
R₆ represents hydrogen, halogen, CN, C₁₋₆ alkyl, C₃₋₇ cycloalkyl, OR₁, CF₃, COOR₁, S(O)ₙR₁; S(O)₂NR₁ₐR₂; (CH₂)ₘC₅₋₁₀ heterocyclyl, —NS(O)₂NR₁ₐR₂, or is absent when D is nitrogen said alkyl and heterocyclyl optionally substituted with 1 to 3 groups of halogen, C₁₋₆ alkyl, (CH₂)ₘC₃₋₇ cycloalkyl, CN, (CH₂)ₘOR₁, CF₃, OCF₃, —NHC(O)R₁, CH(O), (CH₂)ₘC₆₋₁₀ aryl, C(O)C₆₋₁₀ aryl, (CH₂)ₘN(R₁)₂, C(O)N(R₁)₂, (CH₂)ₘCOOR₁, and (CH₂)ₘS(O)ₙR₁;
R₇ represents hydrogen, halogen, C₁₋₆ alkyl, C(O)OR₁, —C(CH₃)₂OH, —CH═CHC(O)N(R₁)₂, (CH₂)ₘC₃₋₇ cycloalkyl, CN, OR₁, CF₃, S(O)ₙR₁, CONR₉R¹⁰, NR₁CONR₁R₉, (CH₂)ₘC₆₋₁₀ aryl, (CH₂)ₘC₅₋₁₀ heterocyclyl, or is absent when B is nitrogen said alkyl, aryl and heterocyclyl optionally substituted with 1 to 3 groups of halogen, C₁₋₆ alkyl, (CH₂)ₘC₃₋₇ cycloalkyl, CN, (CH₂)ₘOR₁, CF₃, OCF₃, —NHC(O)R₁, CH(O), (CH₂)ₘC₆₋₁₀ aryl, C(O)C₆₋₁₀ aryl, (CH₂)ₘN(R₁)₂, C(O)N(R₁)₂, (CH₂)ₘCOOR₁, and (CH₂)ₘS(O)ₙR₁;
R₉ and R¹⁰ independently represent hydrogen, (CH₂)ₘ aryl, C₂-C₆ alkenyl, C₂-C₆ alkynyl, (CH₂)ₘ heterocyclyl, C₃-C₆ cycloalkyl, SO₂R⁷, and (C═O)N(R₁)₂, said alkyl, cycloalkyl, aryl, heterocylyl, alkenyl, and alkynyl optionally substituted with one or more substituents selected from halogen, C₁₋₆ alkyl, CN, CF₃, (CH₂)ₘN(R₁)₂, (CH₂)ₘOR₁, (CH₂)ₘCOOR₁, (CH₂)ₘS(O)ₙR₁;
R⁹ and R¹⁰ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, (CH₂)ₘOR₁, CN, CF₃, N(R₁)₂, COOR₁.
n is an integer from 0 to 2;
m is an integer from 0 to 6; and
x is an integer from 1 to 3.

An embodiment of this invention is realized when R₁ and R₁a together with the nitrogen that R₁a is attached form a monocyclic or bicyclic heterocycle, unsaturated or saturated, with 4-7 members in each ring and optionally containing in addition to the nitrogen, one or two additional heteroatoms selected from N, O and S, said monocylcic or bicyclic heterocycle optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and all other variables are as described herein. A sub-embodiment of this invention is realized when the heterocyclic group formed is a saturated ring. A sub-embodiment of this invention is realized when the ring is piperidine. Another sub-embodiment of this invention is realized when the ring is pyrrolidine. Still another sub-embodiment of this invention is realized when the ring is azetidine.

Another embodiment of this invention is realized when R₂ is hydrogen and all other variables are as originally described.

Still another embodiment of this invention is realized when R₃ and R₄ both are hydrogen and all other variables are as originally described.

Yet another embodiment of this invention is realized when R₅ is aryl optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and all other variables are as described herein. A sub-embodiment of this invention is realized when the aryl is phenyl.

Yet another embodiment of this invention is realized when R₅ is heterocyclyl optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and all other variables are as described herein.

Still another embodiment of this invention is realized when R₇ is aryl optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and all other variables are as described herein. A sub-embodiment of this invention is realized when the aryl is phenyl.

Still another embodiment of this invention is realized when R₇ is heterocyclyl optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and all other variables are as described herein. A sub-embodiment of this invention is realized when the heterocyclyl is a C₅₋₁₀ heteroaryl.

Still another embodiment of this invention is realized when D is nitrogen and R₆ is absent.

Still another embodiment of this invention is realized when B is nitrogen and R₇ is absent.

Still another embodiment of this invention is realized when A, B, D, and F are all carbon.

Another embodiment of this invention is realized with the compounds of structural formula II:

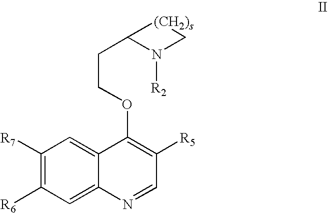

And pharmaceutically acceptable salts, esters, enantiomers, diastereomers or mixtures thereof wherein s is an integer from 1 to 3, R₂ is hydrogen and R₅, R₆ and R₇ are as originally described. A sub-embodiment of this invention is realized when s is 1. Another sub-embodiment is realized when s is 2. Still another sub-embodiment of this invention realized when s is 3. Yet another sub-embodiment of the invention of formula II is realized when R₅ and R₇ both are aryl optionally substituted with one or more substituents selected from halogen, C₁₋₆ alkyl, (CH₂)ₘOR₁, CN, CF₃, (CH₂)ₘN(R₁)₂, NO₂, (CH₂)ₘCOOR₁, and all other variables are as described herein. A sub-embodiment of this invention is realized when the aryl is phenyl. Another sub-embodiment of the invention of formula II is realized when R₇ is a heteroaryl and R₅ is an aryl, both optionally substituted with one or more substituents selected from halogen, C₁₋₆ alkyl, (CH₂)ₘOR₁, CN, CF₃, (CH₂)ₘN(R₁)₂, NO₂, (CH₂)ₘCOOR₁, and all other variables are as described herein.

Still another sub-embodiment of the invention of formula II is represented by the compounds of formula IIa:

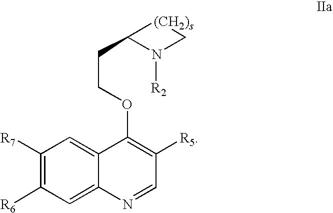

Another embodiment of this invention is realized with the compounds of structural formula III:

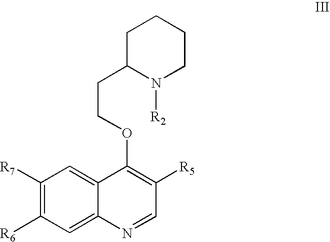

and pharmaceutically acceptable salts, esters, enantiomers, diastereomers or mixtures thereof wherein R₂ is hydrogen and R₅, R₆ and R₇ are as originally described. A sub-embodiment is realized when R₅ and R₇ both are aryl optionally substituted with one or more substituents selected from halogen, C₁₋₆ alkyl, (CH₂)ₘOR₁, CN, CF₃, (CH₂)ₘN(R₁)₂, NO₂, (CH₂)ₘCOOR₁, and all other variables are as described herein. A sub-embodiment of this invention is realized when the aryl is phenyl. Another sub-embodiment of the invention of Formula III is realized when R₇ is a heteroaryl and R₅ is an aryl, both optionally substituted with one or more substituents selected from halogen, C₁₋₆ alkyl, (CH₂)ₘOR₁, CN, CF₃, (CH₂)ₘN(R₁)₂, NO₂, (CH₂)ₘCOOR₁, and all other variables are as described herein.

A sub-embodiment of the invention of formula III is represented by the compound of formula IIIa

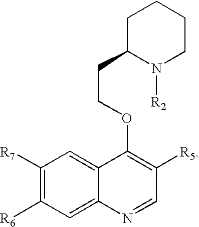

Asymmetric centers may be present in the compounds of the instant invention depending upon the nature of the various substituents on the molecule. Each such asymmetric center will independently produce two optical isomers and it is intended that all of the possible optical isomers and diastereomers in mixture and as pure or partially purified compounds are included within the ambit of this invention. In the case of the asymmetric carbon atom represented in Formula III (designated the R isomer), it has been found that these compounds are more active and selective as somatostatin agonists

Compounds of this invention are:
7-chloro-3-(3,5-dimethylphenyl)-6-(1-methyl-1H-pyrazol-4-yl)-4-{2-[(2R)-piperidin-2-yl]ethoxy}quinoline;
3-[4-(3-aminopropoxy)-7-chloro-3-(3,5-dimethylphenyl)quinolin-6-yl]benzamide;
3-(3,5-dimethylphenyl)-6-(1H-indazol-5-yl)-4-{2-[(2R)-piperidin-2-yl]ethoxy}quinoline;
2-[7-chloro-3-(3,5-dimethylphenyl)-4-(2-piperidin-2-ylethoxy)quinolin-6-yl]propan-2-ol;
7-chloro-3-(3,5-dimethylphenyl)-6-(1H-indazol-5-yl)-4-{2-[(2R)-piperidin-2-yl]ethoxy}quinoline;
7-chloro-3-(3,5-dimethylphenyl)-6-(1-methyl-1H-pyrazol-4-yl)-4-{2-[(2S)-piperidin-2-yl]ethoxy}-quinoline
3-(7-chloro-3-(3,5-dimethylphenyl)-4-{2-[(2R)-piperidin-2-yl]ethoxy}quinolin-6-yl)benzamide;
4-(7-chloro-3-(3,5-dimethylphenyl)-4-{2-[(2R)-piperidin-2-yl]ethoxy}quinolin-6-yl)benzamide;
3-(7-chloro-3-(3,5-dimethylphenyl)-4-{2-[(2R)-piperidin-2-yl]ethoxy}quinolin-6-yl)phenol;
7-chloro-3-(3,5-dimethylphenyl)-6-(2-methoxypyrimidin-5-yl)-4-{2-[(2R)-piperidin-2-yl]ethoxy}quinoline;
7-chloro-6-(2-chloropyridin-4-yl)-3-(3,5-dimethylphenyl)-4-{2-[(2R)-piperidin-2-yl]ethoxy}quinoline;
{3-[4-(3-aminopropoxy)-7-chloro-3-(3,5-dimethylphenyl)quinolin-6-yl]phenyl}methanol;
7-chloro-3-(3,5-dimethylphenyl)-6-(4-fluorophenyl)-4-(2-piperidin-2-ylethoxy)quinoline;
7-chloro-3-(3,5-dimethylphenyl)-4-(2-piperidin-2-ylethoxy)-6-(1,3-thiazol-2-yl)quinoline;
7-chloro-3-(3,5-dimethylphenyl)-4-{2-[(2R)-piperidin-2-yl]ethoxy}-6-(1H-pyrazol-5-yl)quinoline;
(3-{[7-chloro-3-(3,5-dimethylphenyl)-6-(1-methyl-1H-pyrazol-3-yl)quinolin-4-yl]oxy}propyl)amine;
7-chloro-3-(3,5-dimethylphenyl)-6-(2-fluoropyridin-4-yl)-4-{2-[(2R)-piperidin-2-yl]ethoxy}quinoline;
6-bromo-7-chloro-3-(3,5-dimethylphenyl)-4-{2-[(2R)-piperidin-2-yl]ethoxy}quinoline;
4-(7-chloro-3-(3,5-dimethylphenyl)-4-{2-[(2R)-piperidin-2-yl]ethoxy}quinolin-6-yl)phenol;
4,4′-(7-chloro-4-{2-[(2R)-piperidin-2-yl]ethoxy}quinoline-3,6-diyl)diphenol;
[3-(7-chloro-3-(3,5-dimethylphenyl)-4-{2-[(2R)-piperidin-2-yl]ethoxy}quinolin-6-yl)phenyl]methanol;
5-(7-chloro-3-(3,5-dimethylphenyl)-4-{2-[(2R)-piperidin-2-yl]ethoxy}quinolin-6-yl)pyrimidine-2,4-diol;
[4-(7-chloro-3-(3,5-dimethylphenyl)-4-{2-[(2R)-piperidin-2-yl]ethoxy}quinolin-6-yl)phenyl]methanol;
methyl 7-chloro-3-(3,5-dimethylphenyl)-4-(2-piperidin-2-ylethoxy)quinoline-6-carboxylate;
7-chloro-3-(3,5-dimethylphenyl)-6-(1H-indazol-6-yl)-4-(2-piperidin-2-ylethoxy)quinoline;
3,3′-(7-chloro-4-{2-[(2R)-piperidin-2-yl]ethoxy}quinoline-3,6-diyl)dibenzamide;
7-chloro-3-(3,5-dimethylphenyl)-4-{2-[(2R)-piperidin-2-yl]ethoxy}-6-pyridin-4-ylquinoline;
3-(3,5-dimethylphenyl)-4-(2-piperidin-2-ylethoxy)-6,7-di-1,3-thiazol-2-ylquinoline;
7-chloro-3-(3,5-dimethylphenyl)-6-(6-fluoropyridin-3-yl)-4-{2-[(2R)-piperidin-2-yl]ethoxy}quinoline;
7-chloro-3,6-bis(6-fluoropyridin-3-yl)-4-{2-[(2R)-piperidin-2-yl]ethoxy}quinoline;
7-chloro-6-(6-chloropyridin-3-yl)-3-(3,5-dimethylphenyl)-4-{2-[(2R)-piperidin-2-yl]ethoxy}quinoline;
4-(2-azetidin-2-ylethoxy)-7-chloro-3-(3,5-dimethylphenyl)-6-(1H-indazol-5-yl)quinoline;
7-chloro-3-(3,5-dimethylphenyl)-4-{2-[(2S)-piperidin-2-yl]ethoxy}-6-pyridin-3-ylquinoline;
4-(2-azetidin-2-ylethoxy)-7-chloro-3-(3,5-dimethylphenyl)-6-(1-methyl-1H-pyrazol-3-yl)quinoline;
7-chloro-3-(3,5-dimethylphenyl)-4-{2-[(2R)-piperidin-2-yl]ethoxy}-6-(1H-pyrazol-4-yl)quinoline;
3-(7-chloro-3-(3,5-dimethylphenyl)-4-{2-[(2R)-piperidin-2-yl]ethoxy}quinolin-6-yl)-N-methylbenzamide;
7-chloro-3-(3,5-dimethylphenyl)-6-(5-methoxypyridin-3-yl)-4-{2-[(2R)-piperidin-2-yl]ethoxy}quinoline;
3,3′-(4-{2-[(2R)-piperidin-2-yl]ethoxy}quinoline-3,6-diyl)dibenzamide;
3,3′-(7-chloro-4-{2-[(2R)-piperidin-2-yl]ethoxy}quinoline-3,6-diyl)diphenol;
4-(2-azetidin-2-ylethoxy)-7-chloro-3-(3,5-dimethylphenyl)-6-(6-fluoropyridin-3-yl)quinoline;
7-chloro-3,6-bis(4-fluorophenyl)-4-{2-[(2R)-piperidin-2-yl]ethoxy}quinoline;
4-(2-azetidin-2-ylethoxy)-7-chloro-3-(3,5-dimethylphenyl)-6-pyridin-4-ylquinoline;
(2E)-3-(7-chloro-3-(3,5-dimethylphenyl)-4-{2-[(2R)-piperidin-2-yl]ethoxy}quinolin-6-yl)acrylamide;
5-[4-(2-azetidin-2-ylethoxy)-7-chloro-3-(3,5-dimethylphenyl)quinolin-6-yl]pyridin-2-amine;
(3-{[7-chloro-3-(3,5-dimethylphenyl)-6-(1H-indazol-5-yl)quinolin-4-yl]oxy}propyl)amine;
4-(2-azetidin-2-ylethoxy)-7-chloro-3-(3,5-dimethylphenyl)-6-pyridin-3-ylquinoline;
3-[4-(2-azetidin-2-ylethoxy)-7-chloro-3-(3,5-dimethylphenyl)quinolin-6-yl]phenol;
3,6-bis(1-methyl-1H-pyrazol-4-yl)-4-{2-[(2S)-piperidin-2-yl]ethoxy}quinoline;
3-[4-(2-azetidin-2-ylethoxy)-7-chloro-3-(3,5-dimethylphenyl)quinolin-6-yl]benzamide;
5-[4-(2-azetidin-2-ylethoxy)-7-chloro-3-(3,5-dimethylphenyl)quinolin-6-yl]pyrimidine-2,4-diol;
3,6-di-1H-indazol-5-yl-4-{2-[(2R)-piperidin-2-yl]ethoxy}quinoline;
6-(4-fluorophenyl)-3-phenyl-4-(2-piperidin-2-ylethoxy)quinoline;
4-(2-azetidin-2-ylethoxy)-7-chloro-3-(3,5-dimethylphenyl)-6-pyrimidin-5-ylquinoline;
6-(3-methylphenyl)-3-phenyl-4-(2-piperidin-2-ylethoxy)quinoline;
{[5-(7-chloro-3-(3,5-dimethylphenyl)-4-{2-[(2R)-piperidin-2-yl]ethoxy}quinolin-6-yl)-3-fluoropyridin-2-yl]methyl}amine;
6-(3-fluorophenyl)-3-phenyl-4-(2-piperidin-2-ylethoxy)quinoline;
7-chloro-3-(3,5-dimethylphenyl)-4-{2-[(2R)-piperidin-2-yl]ethoxy}quinoline;
6-(4-methoxyphenyl)-3-phenyl-4-(2-piperidin-2-ylethoxy)quinoline;
3,6-bis(4-fluorophenyl)-4-{2-[(2R)-piperidin-2-yl]ethoxy}quinoline;
3-phenyl-4-(2-piperidin-2-ylethoxy)-6-(4-propylphenyl)quinoline;
7-chloro-3-(3,5-dimethylphenyl)-4-{2-[(2S)-piperidin-2-yl]ethoxy}-6-(1H-pyrazol-4-yl)quinoline;
6-(2-naphthyl)-3-phenyl-4-(2-piperidin-2-ylethoxy)quinoline;
3,6-bis(6-fluoropyridin-3-yl)-4-{2-[(2R)-piperidin-2-yl]ethoxy}quinoline;
6-biphenyl-4-yl-3-phenyl-4-(2-piperidin-2-ylethoxy)quinoline;
(3-{[7-chloro-3-(3,5-dimethylphenyl)-6-pyrimidin-5-ylquinolin-4-yl]oxy}propyl)amine;
phenyl {4-[3-phenyl-4-(2-piperidin-2-ylethoxy)quinolin-6-yl]phenyl}methanone;
3-[4-(2-azetidin-2-ylethoxy)-7-chloro-3-(3,5-dimethylphenyl)quinolin-6-yl]benzaldehyde;
6-(6-methoxypyridin-3-yl)-3-phenyl-4-(2-piperidin-2-ylethoxy)quinoline;
7-chloro-3,6-bis(1-methyl-1H-pyrazol-4-yl)-4-{2-[(2R)-piperidin-2-yl]ethoxy}quinoline;
6-isoquinolin-4-yl-3-phenyl-4-(2-piperidin-2-ylethoxy)quinoline;
{3-[4-(2-azetidin-2-ylethoxy)-7-chloro-3-(3,5-dimethylphenyl)quinolin-6-yl]phenyl}methanol;
6-[4-(methylsulfonyl)phenyl]-3-phenyl-4-(2-piperidin-2-ylethoxy)quinoline;
5-[4-(3-aminopropoxy)-7-chloro-3-(3,5-dimethylphenyl)quinolin-6-yl]pyridin-2-amine;
4-[7-chloro-3-(3,5-dimethylphenyl)-4-(2-piperidin-2-ylethoxy)quinolin-6-yl]benzonitrile;
4,4′-(4-{2-[(2R)-piperidin-2-yl]ethoxy}quinoline-3,6-diyl)diphenol;
7-chloro-3-(3,5-dimethylphenyl)-6-(3-methoxyphenyl)-4-(2-piperidin-2-ylethoxy)quinoline;
(2E)-3-(7-chloro-3-(3,5-dimethylphenyl)-4-{2-[(2S)-piperidin-2-yl]ethoxy}quinolin-6-yl)acrylamide;
{3-[7-chloro-3-(3,5-dimethylphenyl)-4-(2-piperidin-2-ylethoxy)quinolin-6-yl]phenyl}methanol;
4-(2-azetidin-2-ylethoxy)-7-chloro-3-(3,5-dimethylphenyl)-6-(1H-pyrazol-3-yl)quinoline;
7-chloro-6-(3,4-difluorophenyl)-3-(3,5-dimethylphenyl)-4-(2-piperidin-2-ylethoxy)quinoline;
5-[4-(3-aminopropoxy)-7-chloro-3-(3,5-dimethylphenyl)quinolin-6-yl]pyrimidine-2,4-diol;
7-chloro-3-(3,5-dimethylphenyl)-6-(2-methoxy-5-methylphenyl)-4-(2-piperidin-2-ylethoxy)quinoline;
4-(2-azetidin-2-ylethoxy)-6-bromo-7-chloro-3-(3,5-dimethylphenyl)quinoline;
7-chloro-3-(3,5-dimethylphenyl)-6-(2-fluoro-3-methoxyphenyl)-4-(2-piperidin-2-ylethoxy)quinoline;
5-[4-(2-azetidin-2-ylethoxy)-7-chloro-3-(3,5-dimethylphenyl)quinolin-6-yl]pyridin-2-ol;
6-(1-benzothien-3-yl)-7-chloro-3-(3,5-dimethylphenyl)-4-(2-piperidin-2-ylethoxy)quinoline;
7-chloro-3-(3,5-dimethylphenyl)-4-piperidin-1-ylquinoline;
N-{3-[7-chloro-3-(3,5-dimethylphenyl)-4-(2-piperidin-2-ylethoxy)quinolin-6-yl]phenyl}acetamide;
6-(2-methylphenyl)-3-phenyl-4-(2-piperidin-2-ylethoxy)quinoline;
7-chloro-6-(3,4-dimethoxyphenyl)-3-(3,5-dimethylphenyl)-4-(2-piperidin-2-ylethoxy)quinoline;
6-(2-fluorophenyl)-3-phenyl-4-(2-piperidin-2-ylethoxy)quinoline;
7-chloro-6-(2,6-dimethoxyphenyl)-3-(3,5-dimethylphenyl)-4-(2-piperidin-2-ylethoxy)quinoline;
4-[3-phenyl-4-(2-piperidin-2-ylethoxy)quinolin-6-yl]benzonitrile;
5-[7-chloro-3-(3,5-dimethylphenyl)-4-(2-piperidin-2-ylethoxy)quinolin-6-yl]-2-methoxyphenol;
6-(4-chlorophenyl)-3-phenyl-4-(2-piperidin-2-ylethoxy)quinoline;
3-[7-chloro-3-(3,5-dimethylphenyl)-4-(2-piperidin-2-ylethoxy)quinolin-6-yl]phenol;
1-{4-[3-phenyl-4-(2-piperidin-2-ylethoxy)quinolin-6-yl]phenyl}ethanone;
7-chloro-3-(3,5-dimethylphenyl)-4-(2-piperidin-2-ylethoxy)-6-pyridin-3-ylquinoline;
3-phenyl-4-(2-piperidin-2-ylethoxy)-6-[4-(trifluoromethyl)phenyl]quinoline;
7-chloro-3-(3,5-dimethylphenyl)-4-(2-piperidin-2-ylethoxy)-6-(1H-pyrazol-4-yl)quinoline;
3-phenyl-4-(2-piperidin-2-ylethoxy)-6-[4-(trifluoromethoxy)phenyl]quinoline;
7-chloro-3-(3,5-dimethylphenyl)-6-(1-methyl-1H-pyrazol-4-yl)-4-(2-piperidin-2-ylethoxy)quinoline;
3-phenyl-4-(2-piperidin-2-ylethoxy)-6-pyridin-3-ylquinoline;
[(7-chloro-4-{2-[(2R)-piperidin-2-yl]ethoxy}quinoline-3,6-diyl)bis(3,1-phenylene)]dimethanol;
6-cyclohex-1-en-1-yl-3-phenyl-4-(2-piperidin-2-ylethoxy)quinoline;
3-(3,5-dimethylphenyl)-6-(4-fluorophenyl)-4-(2-piperidin-2-ylethoxy)quinoline;
6-(1-naphthyl)-3-phenyl-4-(2-piperidin-2-ylethoxy)quinoline;
3-(3,5-dimethylphenyl)-6-(1-methyl-1H-pyrazol-4-yl)-4-{2-[(2R)-piperidin-2-yl]ethoxy}quinoline;
3-phenyl-4-(2-piperidin-2-ylethoxy)-6,8′-biquinoline;
7-chloro-3-(3,5-dimethylphenyl)-6-(2-methoxypyridin-3-yl)-4-{2-[(2R)-piperidin-2-yl]ethoxy}quinoline;
2-[7-chloro-3-(3,5-dimethylphenyl)-4-(2-piperidin-2-ylethoxy)quinolin-6-yl]benzonitrile;
3-[4-(3-aminopropoxy)-7-chloro-3-(3,5-dimethylphenyl)quinolin-6-yl]phenol,
{4-[7-chloro-3-(3,5-dimethylphenyl)-4-(2-piperidin-2-ylethoxy)quinolin-6-yl]phenyl}methanol;
7-chloro-3-(3,5-dimethylphenyl)-6-(1H-indol-5-yl)-4-{2-[(2R)-piperidin-2-yl]ethoxy}quinoline;
7-chloro-6-(2,4-difluorophenyl)-3-(3,5-dimethylphenyl)-4-(2-piperidin-2-ylethoxy)quinoline;
7-chloro-3-(3,5-dimethylphenyl)-6-(2-fluoropyridin-3-yl)-4-{2-[(2R)-piperidin-2-yl]ethoxy}quinoline;
{4-[7-chloro-3-(3,5-dimethylphenyl)-4-(2-piperidin-2-ylethoxy)quinolin-6-yl]phenyl}dimethylamine;
7-chloro-3-(3,5-dimethylphenyl)-4-{2-[(2R)-piperidin-2-yl]ethoxy}-6-pyrimidin-4-ylquinoline;
7-chloro-3-(3,5-dimethylphenyl)-6-(3-fluoro-4-methoxyphenyl)-4-(2-piperidin-2-ylethoxy)quinoline;
[(4-{2-[(2R)-piperidin-2-yl]ethoxy}quinoline-3,6-diyl)bis(3,1-phenylene)]dimethanol;
7-chloro-3-(3,5-dimethylphenyl)-6-(2-fluoro-5-methoxyphenyl)-4-(2-piperidin-2-ylethoxy)quinoline;
3,3′-(4-{2-[(2R)-piperidin-2-yl]ethoxy}quinoline-3,6-diyl)diphenol;
N-{4-[7-chloro-3-(3,5-dimethylphenyl)-4-(2-piperidin-2-ylethoxy)quinolin-6-yl]phenyl}acetamide;
7-chloro-6-(6-chloropyrazin-2-yl)-3-(3,5-dimethylphenyl)-4-{2-[(2R)-piperidin-2-yl]ethoxy}quinoline;
7-chloro-6-(2,5-dimethoxyphenyl)-3-(3,5-dimethylphenyl)-4-(2-piperidin-2-ylethoxy)quinoline;
5-(7-chloro-3-(3,5-dimethylphenyl)-4-{2-[(2R)-piperidin-2-yl]ethoxy}quinolin-6-yl)pyridin-2-ol;
7-chloro-6-(2,4-dimethoxyphenyl)-3-(3,5-dimethylphenyl)-4-(2-piperidin-2-ylethoxy)quinoline;
7-chloro-3-(3,5-dimethylphenyl)-6-(5-fluoro-6-methylpyridin-2-yl)-4-{2-[(2R)-piperidin-2-yl]ethoxy}quinoline;
7-chloro-6-(2,3-dimethoxyphenyl)-3-(3,5-dimethylphenyl)-4-(2-piperidin-2-ylethoxy)quinoline;
5-(7-chloro-3-(3,5-dimethylphenyl)-4-{2-[(2R)-piperidin-2-yl]ethoxy}quinolin-6-yl)pyridin-2-amine;
4-[7-chloro-3-(3,5-dimethylphenyl)-4-(2-piperidin-2-ylethoxy)quinolin-6-yl]phenol;
7-chloro-3-(3,5-dimethylphenyl)-6-(6-methoxypyridin-3-yl)-4-{2-[(2R)-piperidin-2-yl]ethoxy}quinoline;
7-chloro-3-(3,5-dimethylphenyl)-6-(4-methoxypyridin-3-yl)-4-{2-[(2R)-piperidin-2-yl]ethoxy}quinoline;
[3-(7-chloro-3-(3,5-dimethylphenyl)-4-{2-[(2R)-piperidin-2-yl]ethoxy}quinolin-6-yl)benzyl]amine;
7-chloro-3,6-di-1H-indazol-5-yl-4-{2-[(2R)-piperidin-2-yl]ethoxy}quinoline or
   their pharmaceutically acceptable salts, esters, enantiomers, diastereomers or mixtures thereof.

The invention is described herein in detail using the terms defined below unless otherwise specified.

The compounds of the present invention may contain one or more asymmetric carbon atoms and may exist in racemic and optically active forms. All of these compounds are contemplated to be within the scope of the present invention. Therefore, where a compound is chiral, the separate enantiomers, substantially free of the other, are included within the scope of the invention; further included are all mixtures of the two enantiomers. Also included within the scope of the invention are polymorphs and hydrates of the compounds of the instant invention. (See E. L. Eliel and S. H. Wilen Stereochemistry of Carbon Compounds (John Wiley and Sons, New York 1994), in particular pages 1119-1190).

In addition, the compounds disclosed herein may exist as tautomers and both tautomeric forms are intended to be encompassed by the scope of the invention, even though only one tautomeric structure is depicted. For example, any claim to compound C or D below is understood to include tautomeric structure D or C, and vice versa, as well as mixtures thereof.

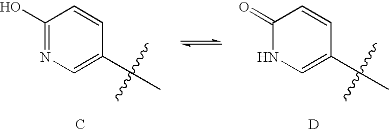

When any variable (e.g. aryl, heterocycle, R₄, R₁ etc.) occurs more than one tine in any constituent, its definition on each occurrence is independent at every other occurrence. Also, combinations of substituents/or variables are permissible only if such combinations result in stable compounds.

The term “alkyl” refers to a monovalent alkane (hydrocarbon) derived radical containing from 1 to 15 carbon atoms unless otherwise defined. It may be straight or branched. Preferred alkyl groups include lower alkyls which have from 1 to 6 carbon atoms such as methyl, ethyl, propyl, isopropyl, butyl and t-butyl. When substituted, alkyl groups may be substituted with up to 5 substituent groups, selected from the groups as herein defined, at any available point of attachment. When the alkyl group is said to be substituted with an alkyl group, this is used interchangeably with “branched alkyl group”.

Cycloalkyl is a species of alkyl containing from 3 to 15 carbon atoms, without alternating or resonating double bonds between carbon atoms. It may contain from 1 to 4 rings which are fused. Preferred cycloalkyl groups are cyclopropyl, cyclobutyl, cyclopentyl and cyclohexyl. When substituted, cycloalkyl groups may be substituted with up to 3 substituents which are defined herein by the definition of alkyl.

The term “alkoxy” refers to those hydrocarbon groups having an oxygen bridge and being in either a straight or branched configuration and if two or more carbon atoms in length, they may include a double or a triple bond. Exemplary of such alkoxy groups are methoxy, ethoxy, propoxy, isopropoxy, butoxy, isobutoxy, tertiary butoxy, pentoxy, isopentoxy, hexoxy, isohexoxy allyloxy, propargyloxy, and the like.

“Halogen” or “halo” as used herein means fluoro, chloro, bromo and iodo.

The term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl. Preferably, alkenyl is C₂-C₆ alkenyl.

Preferably, alkynyl is C₂-C₆ alkynyl.

As used herein, “aryl” is intended to mean any stable monocyclic or bicyclic carbon ring of up to 7 members in each ring, wherein at least one ring is aromatic. Examples of such aryl elements include phenyl, naphthyl, tetrahydronaphthyl, indanyl, biphenyl, phenanthryl, anthryl or acenaphthyl.

The term heterocyclyl, heterocycle or heterocyclic, as used herein, represents a stable 5- to 7-membered monocyclic or stable 8- to 11-membered bicyclic heterocyclic ring which is either saturated or unsaturated, and which consists of carbon atoms and from one to four heteroatoms selected from the group consisting of N, O, and S, and including any bicyclic group in which any of the above-defined heterocyclic rings is fused to a benzene ring. The heterocyclic ring may be attached at any heteroatom or carbon atom which results in the creation of a stable structure. The term heterocyclyl, heterocycle or heterocyclic includes heteroaryl moieties. Examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, 1,3-dioxolanyl, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, and thienyl. An embodiment of the examples of such heterocyclic elements include, but are not limited to, azepinyl, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolidinyl, imidazolinyl, imidazolyl, indolinyl, indolyl, isochromanyl, isoindolinyl, isoquinolinyl, isothiazolidinyl, isothiazolyl, isothiazolidinyl, morpholinyl, naphthyridinyl, oxadiazolyl, 2-oxoazepinyl, oxazolyl, 2-oxopiperazinyl, 2-oxopiperidinyl, 2-oxopyrrolidinyl, piperidyl, piperazinyl, pyridyl, 2-pyridinonyl, pyrazinyl, pyrazolidinyl, pyrazolyl, pyridazinyl, pyrimidinyl, pyrrolidinyl, pyrrolyl, quinazolinyl, quinolinyl, quinoxalinyl, tetrahydrofuryl, tetrahydroisoquinolinyl, tetrahydroquinolinyl, thiamorpholinyl, thiamorpholinyl sulfoxide, thiazolyl, thiazolinyl, thienofuryl, thienothienyl, thienyl and triazolyl.

Preferably, heterocycle is selected from 2-azepinonyl, benzimidazolyl, 2-diazapinonyl, imidazolyl, 2-imidazolidinonyl, indolyl, isoquinolinyl, morpholinyl, piperidyl, piperazinyl, pyridyl, pyrrolidinyl, 2-piperidinonyl, 2-pyrimidinonyl, 2-pyrollidinonyl, quinolinyl, tetrahydrofuryl, tetrahydroisoquinolinyl, and thienyl.

As used herein, “heteroaryl” is intended to mean any stable monocyclic or bicyclic carbon ring of up to 7 members in each ring, wherein at least one ring is aromatic and wherein from one to four carbon atoms are replaced by heteroatoms selected from the group consisting of N, O, and S. Examples of such heterocyclic elements include, but are not limited to, benzimidazolyl, benzisoxazolyl, benzofurazanyl, benzopyranyl, benzothiopyranyl, benzofuryl, benzothiazolyl, benzothienyl, benzoxazolyl, chromanyl, cinnolinyl, dihydrobenzofuryl, dihydrobenzothienyl, dihydrobenzothiopyranyl, dihydrobenzothiopyranyl sulfone, furyl, imidazolyl, indolinyl, indolyl, isochromanyl, isoindolinyl, isoquinolinyl, isothiazolyl, naphthyridinyl, oxadiazolyl, pyridyl, pyrazinyl, pyrazolyl, pyridazinyl, pyrimidinyl, pyrrolyl, quinazolinyl, quinolinyl, quinoxalinyl, tetrahydroisoquinolinyl, tetrahydroquinolinyl, thiazolyl, thienofuryl, thienothienyl, thienyl and triazolyl.

The term “pharmacologically effective amount” shall mean that amount of a drug or pharmaceutical agent that will elicit the biological or medical response of a tissue, system, animal or human that is being sought by a researcher or clinician.

The term “substituted” shall be deemed to include multiple degrees of substitution by a named substitutent.

Where multiple substituent moieties are disclosed or claimed, the substituted compound can be independently substituted by one or more of the disclosed or claimed substituent moieties, singlely or plurally.

As used herein, unless otherwise specifically defined, substituted alkyl, substituted cycloalkyl, substituted aroyl, substituted aryl, substituted heteroaroyl, substituted heteroaryl, substituted arylsulfonyl, substituted heteroaryl-sulfonyl and substituted heterocycle include moieties containing from 1 to 3 substituents, substituents in addition to the point of attachment to the rest of the compound. Preferably, such substituents are selected from the group which includes but is not limited to F, Cl, Br, CF₃, NH₂, N(C₁-C₆ alkyl)₂, NO₂, CN, (C₁-C₆ alkyl)O—, (aryl)O—, (C₁-C₆ alkyl)S(O)ₘ—, (C₁-C₆ alkyl)C(O)NH—, H₂N—C(NH)—, (C₁-C₆ alkyl)C(O)—, (C₁-C₆ alkyl)OC(O)—, (C₁-C₆ alkyl)OC(O)NH—, phenyl, pyridyl, imidazolyl, oxazolyl, isoxazolyl, thiazolyl, thienyl, furyl, isothiazolyl and C₁-C₂₀ alkyl, (CH₂)ₙOH, CF₃, (CH₂)ₙC(O)OH, (CH₂)ₙC(O)OC₁₋₆ alkyl, (CH₂)ₙC(O)NR₇R₉, (CH₂)ₙC₅₋₁₀ heterocyclyl, SO₂NR₅R₆, (CH₂)C₆₋₁₀ aryl, N(R)₂, NO₂, CN, (C₁-C₆ alkyl)O—, (aryl)O—, (C₁₋₆ alkyl)S(O)_{0-2 —, C}₁₋₁₂ alkyl, said heterocyclyl, and aryl optionally substituted with 1 to 3 groups selected from the group consisting of (CH₂)ₙOR, (CH₂)ₙN(R)₂, —O—.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

The pharmaceutically acceptable salts of the compounds of this invention include the conventional non-toxic salts as formed, from non-toxic inorganic or organic bases. For example, such conventional non-toxic salts include those derived from inorganic bases such as an alkali or alkaline earth metal hydroxide, e.g., potassium, sodium, lithium, calcium, or magnesium, and the like: and the salts prepared from organic bases such as an amine, e.g., dibenzylethylene-diamine, trimethylamine, piperidine, pyrrolidine, benzylamine and the like, or a quaternary ammonium hydroxide such as tetramethylammonium hydroxide and the like.

The pharmaceutically acceptable salts can be synthesized from the compounds of this invention by conventional chemical methods. Generally, the salts are prepared by reacting the free acid with stoichiometric amounts or with an excess of the desired salt-forming inorganic or organic base in a suitable solvent or various combinations of solvents.

Also included in the invention is a pharmaceutical composition which is comprised of a compound of formula I in combination with a pharmaceutically acceptable carrier.

The invention also includes a method of treating diabetes, cancer, acromegaly, pain, arthritis, inflammatory bowel disease, irritable bowel syndrome and restenosis, which comprises administering to an animal a compound of formula I in an amount which is effective for treating said disease or condition.

The ability of the compounds of the present invention to act as somatostatin agonists makes them useful as pharmacologic agents for mammals, especially for humans, for the treatment and prevention of disorders wherein somatostatin itself or the hormones it regulates may be involved. Examples of such disorders include diabetes, diabetes-related pathologies, including retinopathy, neuropathy and nephropathy, acromegaly, arthritis, cancer, pain, inflammatory bowel disease, irritable bowel syndrome and restenosis.

The instant compounds can also be used in combination with other therapeutic agents such as metformin or other bifuanides, acarbose, sulfonylureas theazolidinediones or other insulin sensitizers including, but not limited to, compounds which function as agonists on peroxisome proliferator-activated receptor gamma (PPAR-gamma), insulin, insulin-like-growth factor I, glucagon-like peptide I-glp-I and available satiety-promoting agents such as dexfenfluramine or leptin. They may also be used in combination with other analgesics, anti-proliferative, anti-inflammatory or anti-angiogenic agents.

The compounds of the present invention can be administered in such oral dosage forms as tablets, capsules (each including timed release and sustained release formulations), pills, powders, granules, elixers, tinctures, suspensions, syrups and emulsions. Likewise, they may also be administered in intraocular, periocular, topical ocular, intravenous (both bolus and infusion), intraperitoneal, subcutaneous or intramuscular form, all using forms well known to those of ordinary skill in the pharmaceutical arts. An effective but non-toxic amount of the compound desired can be employed as a tocolytic agent.

The dosage regimen utilizing the compounds of the present invention is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

Intravenous dosages or oral dosages of the compounds of the present invention, when used for the indicated effects, will range between about 0.001 to 5 mg/kg and 0.1 to 50 mg/kg, respectively. Advantageously, compounds of the present invention may be administered in a single daily dose, or the total daily dosage may be administered in divided doses of two, three or four times daily. The compounds of the present invention may also be formulated to allow slow release from an implant, device or biodegradable sustained release polymers. These slow release formulations and devices may be inserted into the eye, in juxtaposition to the outer surface of the eye or elsewhere in the body. Furthermore, preferred compounds for the present invention can be administered in intranasal form via topical use of suitable intranasal vehicles, or via transdermal routes, using those forms of transdermal skin patches well known to those of ordinary skill in that art. To be administered in the form of a transdermal delivery system, the dosage administration will, of course, be continuous rather than intermittent throughout the dosage regimen.

In the methods of the present invention, the compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, zanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Throughout the instant application, the following abbreviations are used with the following meanings:

BOC, Boc t-butyloxycarbonyl

calc. calculated

DCC Dicyclohexylcarbodiimide

DCM dichloromethane

DIAD diisoproylazodicarboxylate

EI-MS Electron ion-mass spectroscopy

EtOAc ethyl acetate

eq. equivalent(s)

HPLC High pressure liquid chromatography

MHz Megahertz

NMR Nuclear Magnetic Resonance

THF Tetrahydrofuran

The instant compounds can be effective to inhibit the secretion of various hormones and trophic factors in mammals. They may be used to suppress certain endocrine secretions, such as GH, insulin, glucagon and prolactin, in the treatment of disorders such as acromegaly; endocrine tumors such as carcinoids, VIPomas, insulinomas and glucagonomas; or diabetes and diabetes-related pathologies, including retinopathy, neuropathy and nephropathy. The compounds may also be used to suppress exocrine secretions in the pancreas, stomach and intestines, for treatment of disorders such as pancreatitis, fistulas, bleeding ulcers and diarrhea associated with such diseases as AIDS or cholera. Disorders involving autocrine or paracrine secretions of trophic factors such as IGF-1 (as well as some endocrine factors) which may be treated by administration of the instant compounds include cancers of the breast, prostate, and lung (both small cell and non-small cell epidermoids), as well as hepatomas, neuroblastomas, colon and pancreatic adenocarcinomas (ductal type), chondrosarcomas, and melanomas, diabetic retinopathy, and also atherosclerosis associated with vascular grafts and restenosis following angioplasty.

The compounds of the instant invention are further useful to suppress the mediators of neurogenic inflammation (e.g. substance P or the tachykinins), and may be used in the treatment of rheumatoid arthritis; psoriasis; topical inflammation such as is associated with sunburn, eczema, or other sources of itching; inflammatory bowel disease; irritable bowel syndrome; and allergies, including asthma. The compounds can also function as neuromodulators in the central nervous system, with useful applications in the treatment of Alzheimer's disease and other forms of dementia, pain, and headaches. Furthermore, in disorders involving the splanchnic blood flow, including cirrhosis and oesophagal varices, the compounds of the invention can provide cytoprotection.

The preparation of compounds of Formula I of the present invention may be carried out in sequential or convergent synthetic routes. Compounds fused with different aromatic or non aromatic rings and/or bearing additional substituents on these rings are readily prepared by minor modification of the methods herein with procedures known in the art. Syntheses detailing the preparation of the compounds of Formula I are presented in the following reaction schemes.

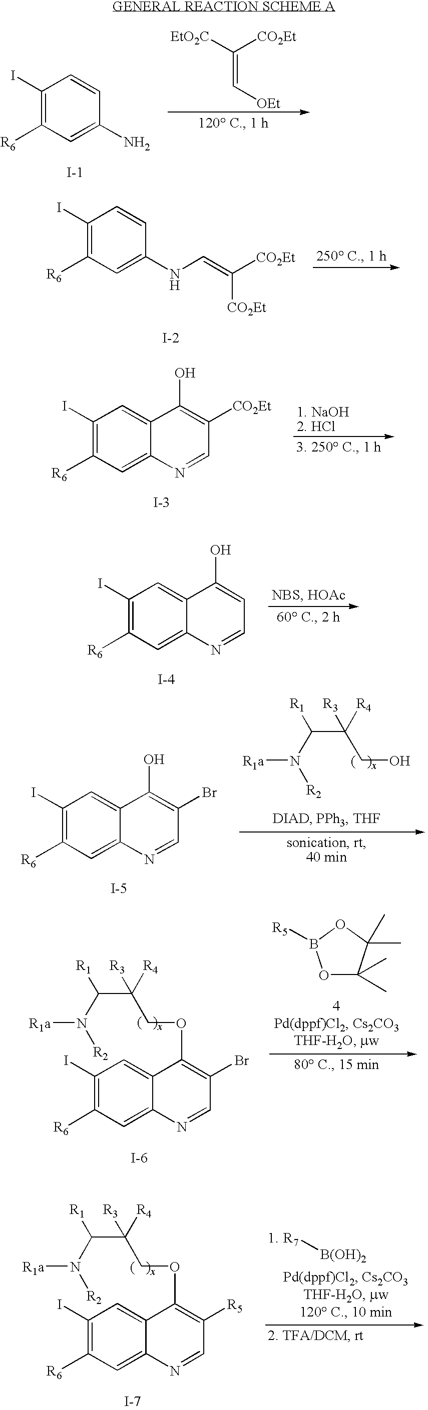

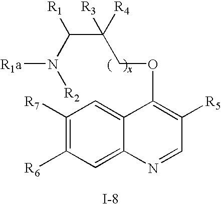

As illustrated in general Reaction Scheme I, a suitably substituted 4-iodoaniline is reacted with 2-ethoxymethylenemalonic acid diethyl ester to provide the enamine, which is cyclized at high temperature to provide the substituted 2-carboethoxyquinoline. After basic hydrolysis and acidification, high temperature induces decarboxylation to furnish the 3-unsubstituted quinoline. This material is brominated, then reacted with an alkyl alcohol bearing a tethered Boc-protected amine under modified Mitsunobu reaction conditions to provide the corresponding ether. Sequential palladium-catalyzed Suzuki reactions furnish the 3,6-diaryl quinoline, which is exposed to trifluoroacetic acid to effect N-Boc deprotection and generate the final material. In this instance, all of aryl boronic acids were commercially available

### Example 1

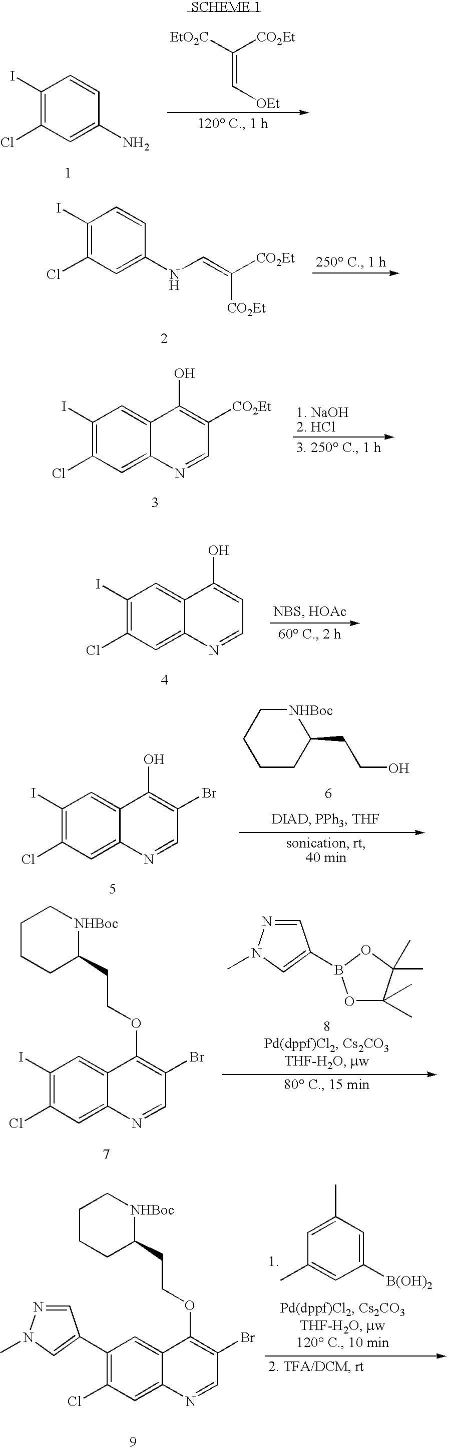

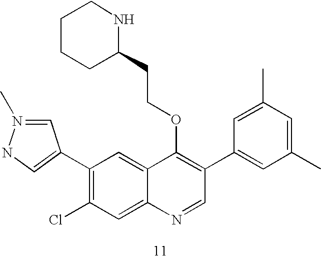

### 7-Chloro-3-(3,5-dimethylphenyl)-6-(1-methyl-1H-pyrazol-4-yl)-4-{2-[(2R)-piperidin-2-yl]ethoxy}quinoline (11)

### Diethyl {[(3-chloro-4-iodophenyl)amino]methylene}malonate (2)

A few boiling chips were added to a mixture of 3-chloro-4-iodo-phenylamine (260.0 g, 1.027 mol) and 2-ethoxymethylene-malonic acid diethyl ester (244.2 g, 1.130 mol) in an open 2-L round-bottomed flask. The mixture was heated at 120□ for 1 h, the evolved ethanol being allowed to escape. The warmed product is used directly in next step (410 g, yield 94.5%). (The anilinoacrylate can be recrystallized from petroleum ether as slender white needles.).

¹H NMR DMSO δ(400 MHz, ppm): 10.55 (d, J=14.0 Hz, 1H), 8.29 (d, J=13.6 Hz, 1H, Ar—H), 7.83 (d, J=8.8 Hz, 1H, Ar—H), 7.67 (d, J=2.4 Hz, 1H, Ar—H), 7.10 (dd, J=8.4, 2.4 Hz, 1H), 4.17 (q, J=7.2 Hz, 2H), 4.09 (q, J=7.2 Hz, 2H), 1.23 (d, J=7.2 Hz, 3H), 1.19 (d, J=7.2 Hz, 3H). LC/MS (ESI) m/e (M⁺+H): 424.0, 426.0.

### Ethyl 7-chloro-4-hydroxy-6-iodoquinoline-3-carboxylate (3)

In a 2-L round-bottomed flask equipped with a condenser 1.5 L of biphenyl ether and compound 2 was heated to vigorous boiling and continued for 1 h. The mixture was cooled, filtered, and the filter was washed with petroleum to obtain the compound 3 (330.0 g, yield 90.4%).

LC/MS (ESI) m/e (M⁺+H): 377.9, 379.9

### 7-Chloro-6-iodoquinolin-4-ol (4)

Compound 3 (312.5 g, 0.827 mol) was mixed with 1 L of 10% aqueous sodium hydroxide, and the mixture was refluxed vigorously until all the solid ester dissolved. The saponification mixture was cooled, and the aqueous solution was separated from any oil that may be present. The solution was acidified to pH=3, the solid was collected and washed with enough water until pH=7, then the solid was washed with two 2.5 L portions of methanol to remove the major impurities and purify the carboxylic acid (281.7 g, yield 97.3%). ¹H NMR DMSO δ (400 MHz, ppm): 14.80 (br, 1H), 12.40 (br, 1H), 8.94 (s, 1H), 8.67 (s, 1H), 7.95 (s, 1H).

LC/MS (ESI) m/e (M⁺+H): 349.9, 351.9

The acid so generated (281.7 g, 0.806 mol) is suspended in 1 L of biphenyl ether in a 2-L flak equipped with a stirrer and a reflux condenser. The mixture was boiling for 1 h, then the mixture was cooled, the solid was collected, and washed with two 2.5 L portions of petroleum, two 2.5 L portions of methanol, two 2.5 L portions of water, 2.5 L portions of acetone to remove the major impurities and purify the final product 4 (241.1 g, yield 97.9%). ¹H NMR DMSO δ(400 MHz, ppm): 11.85 (brs, 1H), 8.48 (s, 1H), 7.93 (d, J=7.6 Hz, 1H), 7.72 (s, 1H), 6.06 (d, J=7.2 Hz, 1H).

### 3-bromo-7-chloro-6-iodoquinolin-4-ol (5)

7-Chloro-6-iodo-quinolin-4-ol 4 (120.0 g, 0.393 mol) in acetic acid (1800 mL) was treated with NBS (70.0 g, 0.393 mol) and the mixture was heated at 60 degrees with stirring for 2 hr, cooled and evaporated. Excess NaHCO3 solution was added and the solid collected and washed with two 2.5 L portions of water, 2.5 L portions of acetone to remove the major impurities and purify the final product 5 (133.0 g, yield 88.1%). ¹H NMR DMSO δ(400 MHz, ppm): 8.52 (s, 1H), 8.49 (s, 1H), 7.74 (s, 1H).

LC/MS (ESI) m/e (M⁺+H): 383.8, 385.8, 387.8

### tert-butyl(2R)-2-{2-[(3-bromo-7-chloro-6-iodoquinolin-4-yl)oxy]ethyl}piperidine-1-carboxylate (7)

Quinolinol 5 (7.69 g, 30.0 mmol), alcohol 2 (4.59 g, 20.0 mmol), PPh₃ (6.30 g, 24.0 mmol), and THF (100 mL) was charged in a 500 mL round flask. The resulting mixture was sealed with a rubber stopper and sonicated for 3 min at rt with shaking, DIAD (4.85 g, 24.0 mmol) was then added through a syringe in 10 min at rt with continues shaking. After addition of DIAD, the reaction was further sonicated for 40 min with shaking. After this period, THF was evaporated and the residue was purified by flash chromatography (EtOAc/hexanes) to give the desired product 7 as a yellow solid (7.39 g, 62%). Analytical LCMS: single peak (214 nm), 4.011 min m/e [M+H]⁺ 595.

### tert-Butyl(2R)-2-(2-{[3-bromo-7-chloro-6-(1-methyl-1H-pyrazol-4-yl)quinolin-4-yl]oxy}ethyl)piperidine-1-carboxylate (9)

A mixture of tert-butyl (2R)-2-{2-[(3-bromo-7-chloro-6-iodoquinolin-4-yl)oxy]ethyl}piperidine-1-carboxylate (7, 696 mg, 1 mmol), 1-methyl-4-(4,4,5,5-tetramethyl-1,3,2-dioxaborolan-2-yl)-1H-pyrazole (8) (315 mg, 1.5 mmole), and Pd(dppf)Cl₂(CH₂Cl₂) (40 mg, 0.05 mmole) in 1M aqueous Cs₂CO₃ (5 mL) and THF (10 mL) solution was microwaved at 80° C. for 15 min. The THF layer was separated and the aqueous layer was extracted with THF (2×5 mL). The combined THF solution was concentrated and the residue was redissolved in DCM (150 mL), washed with brine, dried over Na₂SO₄. Filtration, concentration, and flash chromatograph on silica gel afforded tert-butyl (2R)-2-(2-{[3-bromo-7-chloro-6-(1-methyl-1H-pyrazol-4-yl)quinolin-4-yl]oxy}ethyl)piperidine-1-carboxylate (9) as a slightly solid (452 mg, 83%). Analytical LCMS: single peak (214 nm), 4.115 min m/e [M+H—C₄H₈]⁺ 493.

### 7-Chloro-3-(3,5-dimethylphenyl)-6-(1-methyl-1H-pyrazol-4-yl)-4-{2-[(2R)-piperidin-2-yl]ethoxy}quinoline (11)

A mixture of tert-butyl (2R)-2-(2-{[3-bromo-7-chloro-6-(1-methyl-1H-pyrazol-4-yl)quinolin-4-yl]oxy}ethyl)piperidine-1-carboxylate (9) (55 mg, 0.1 mmole), 3,5-dimethylphenylboronic acid (10) (20 mg, 0.13 mmole), and Pd(dppf)Cl₂(CH₂Cl₂) (4 mg, 0.005 mmole), in 1M aqueous Cs₂CO₃ (0.5 mL)and THF (2 mL) solution was microwaved at 120° C. for 10 min. After cooled to rt, the THF layer was separated and the aqueous layer was extracted with THF (2×2 mL). The combined THF solution was treated with Quadra Pure resin for 2 h to remove Pd. Filtration and concentration afforded a brown residue. This residue was treated with TFA/DCM (1:1, 2 mL) at rt for 1 h. The TFA/DCM solution was concentrated and purified by LCMS to afford the pure 7-chloro-3-(3,5-dimethylphenyl)-6-(1-methyl-1H-pyrazol-4-yl)-4-{2-[(2R)-piperidin-2-yl]ethoxy}quinoline (11) as a slightly yellow solid (TFA salt, 65 mg, 79%). Analytical LCMS: single peak (214 nm), 2.404 min n/e [M+H]⁺ 475. ¹H NMR (600 MHz, CD₃OD): δ 8.81 (s, 1H), 8.35 (s, 1H), 8.21 (S, 1H), 8.19 (S, 1H), 7.92 (S, 1H), 7.26 (S, 2H), 7.19 (S, 1H), 3.96-4.05 (m, 5H), 3.35 (d, J=13.0 Hz, 1H), 3.14-3.20 (m, 1H), 2.92 (dt, J=12.3 Hz, 2.9 Hz, 1H), 2.08-2.15 (m, 1H), 1.73-1.90 (m, 4H), 1.56-1.66 (m, 1H); 1.40-1.49 (m, 1H), 1.26-1.34 (m, 1H); HRMS: calc'd for C₂₈H₃₁ClN₄O (M+H), 475.2259. found 475.2227.

The compounds in Table I below were made using techniques generally known in combination the procedures described in Scheme A, Scheme 1 and Example 1 above and substituting with the appropriate reagents and substrates as required.

TABLE I

| | ESI MS |
|---|---|
| Structure | M + H |
| 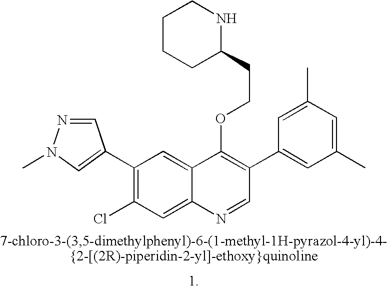 | 476.038 |
| 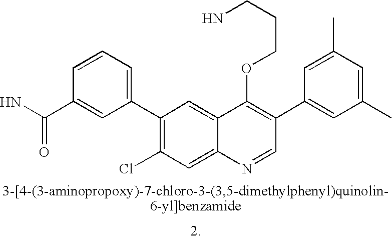 | 460.98 |
| 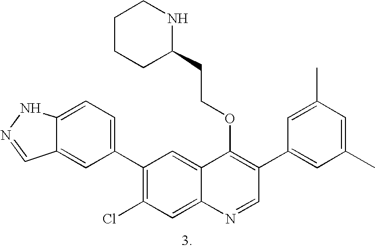 | 512.072 |
| 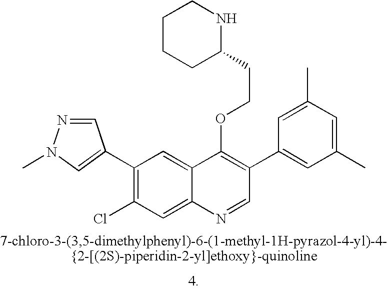 | 476.038 |
| 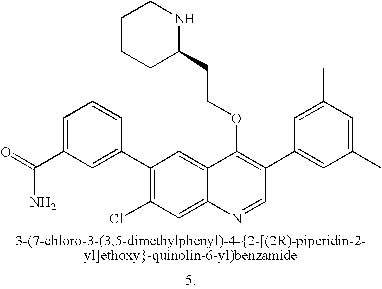 | 515.073 |
| 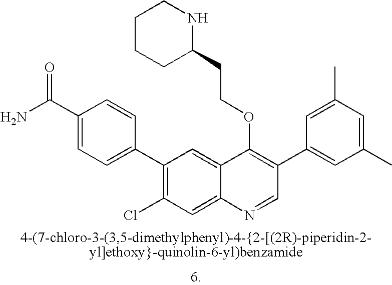 | 515.073 |
| 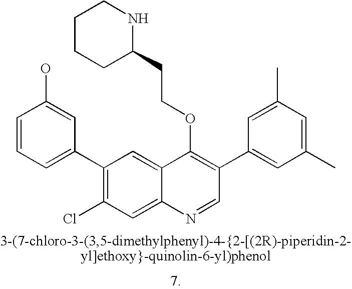 | 488.047 |
| 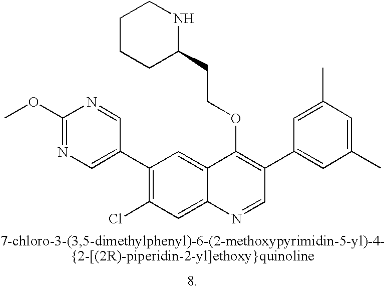 | 504.049 |
| 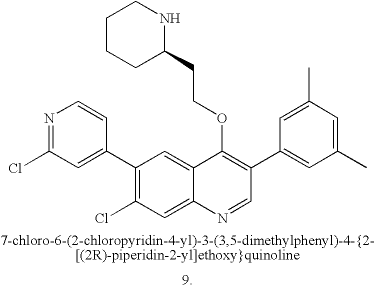 | 507.48 |
| 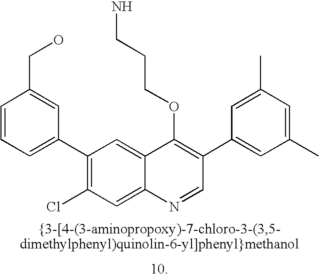 | 447.981 |
| 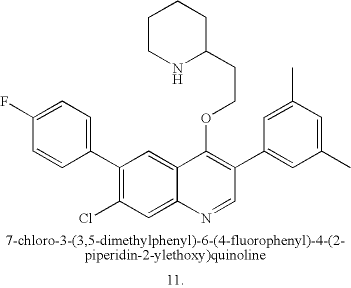 | 490.038 |
| 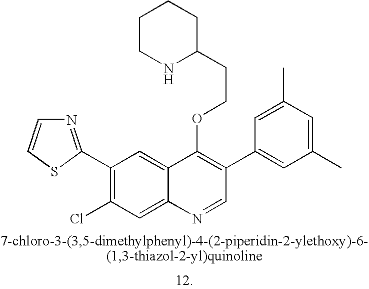 | 479.061 |
| 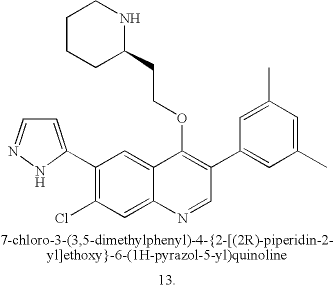 | 462.011 |
| 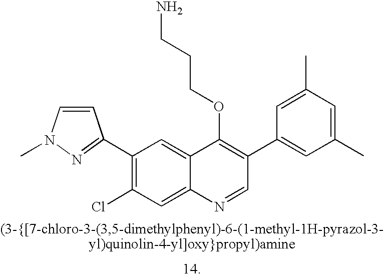 | 421.946 |
| 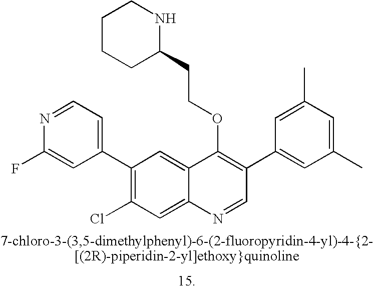 | 491.025 |
| 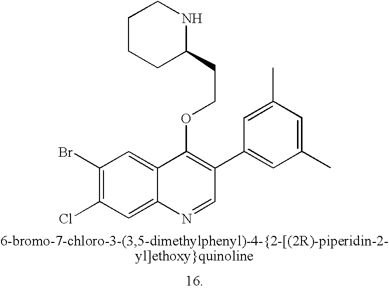 | 474.845 |
| 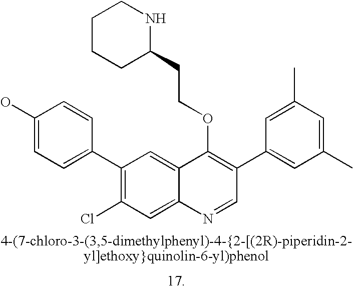 | 488.047 |
| 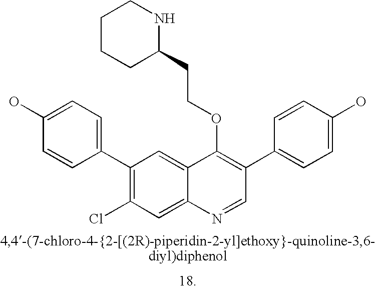 | 475.992 |
| 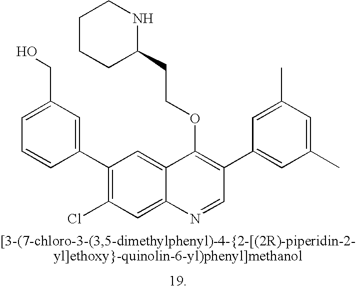 | 502.074 |
| 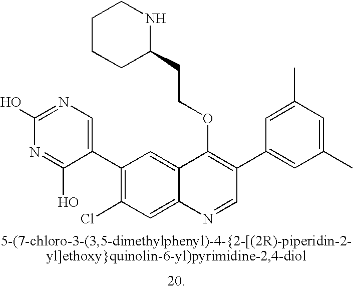 | 506.021 |
| 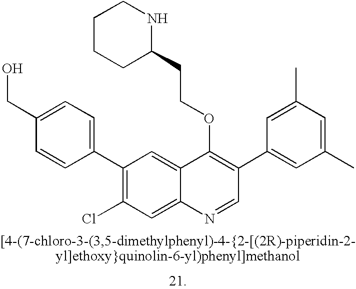 | 502.074 |
| 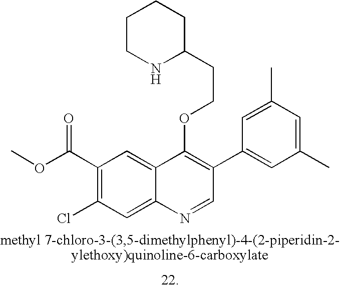 | 453.986 |
| 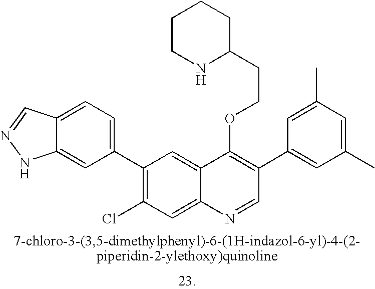 | 512.072 |
| 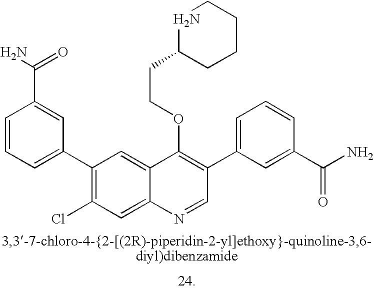 | 530.044 |
| 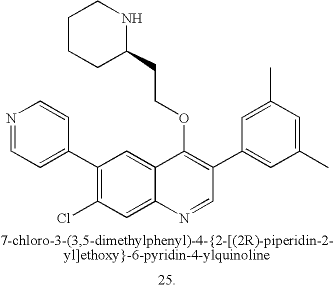 | 473.035 |
| 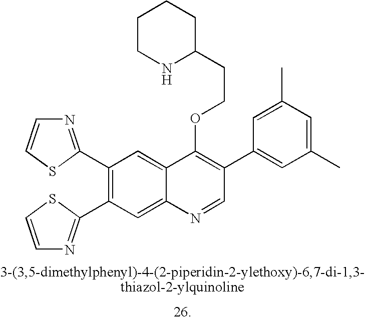 | 527.728 |
| 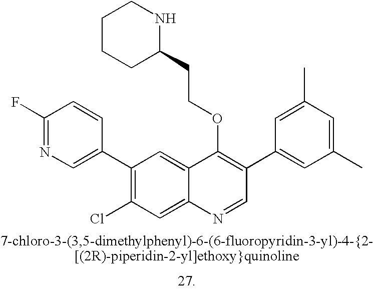 | 491.025 |
| 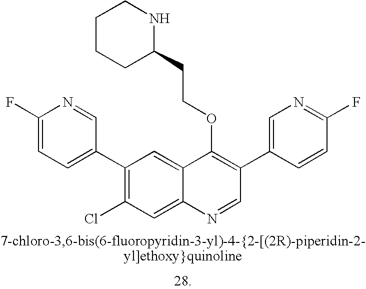 | 481.949 |
| 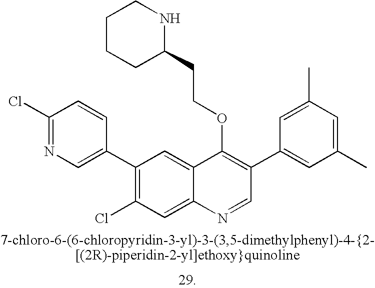 | 507.48 |
| 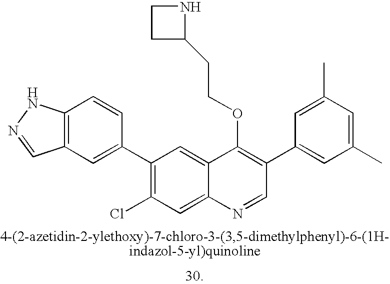 | 484.018 |
| 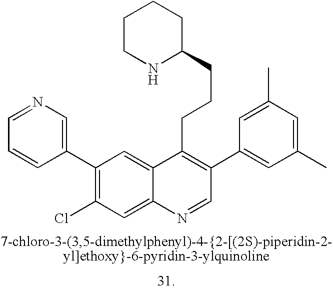 | 473.035 |
| 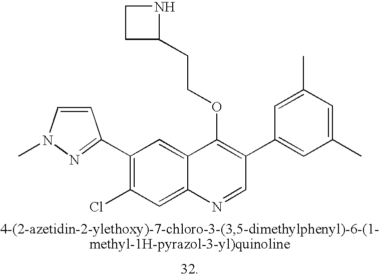 | 447.984 |
| 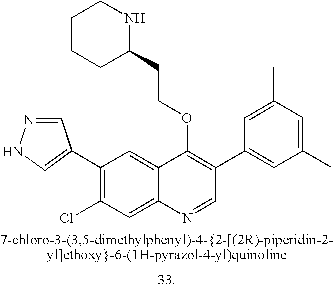 | 462.011 |
| 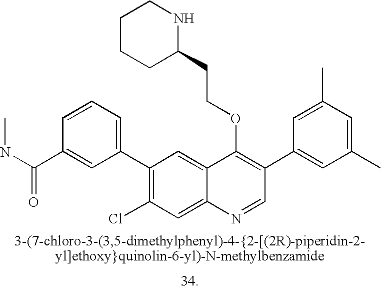 | 529.001 |
| 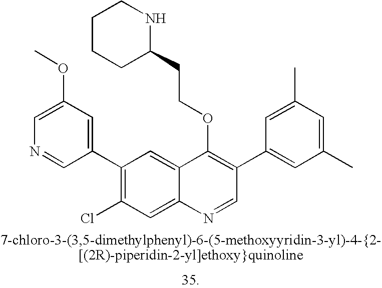 | 503.061 |
| 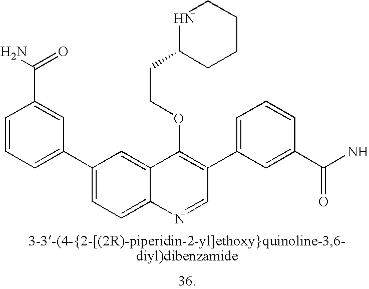 | 495.599 |
| 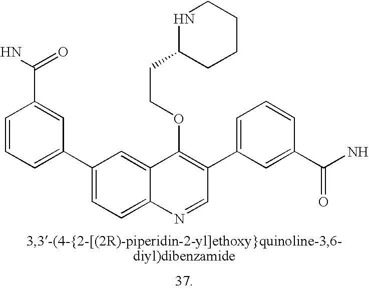 | 475.992 |
| 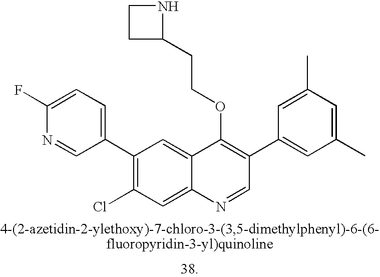 | 462.971 |
| 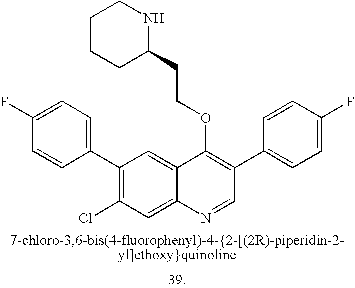 | 479.974 |
| 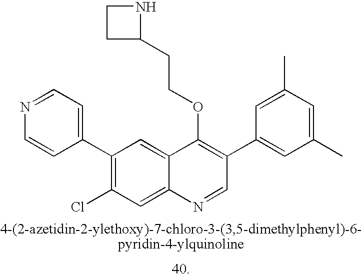 | 444.981 |
| 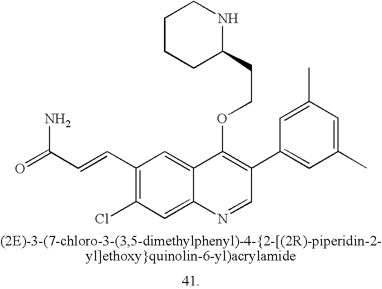 | 465.012 |
| 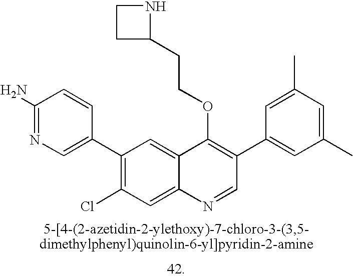 | 459.995 |
| 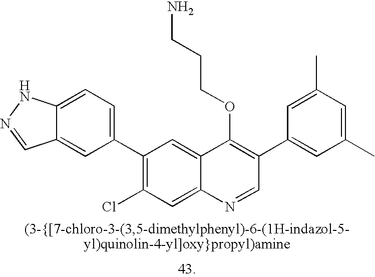 | 457.979 |
| 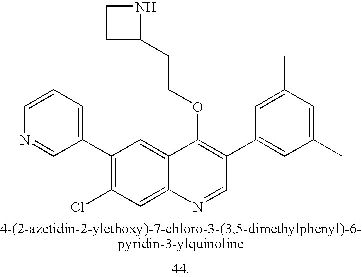 | 444.981 |
| 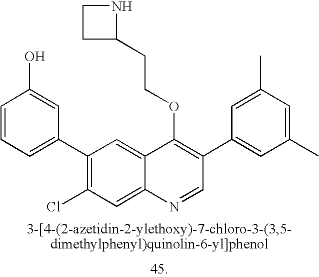 | 459.993 |
| 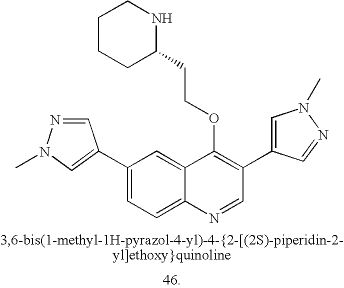 | 417.53 |
| 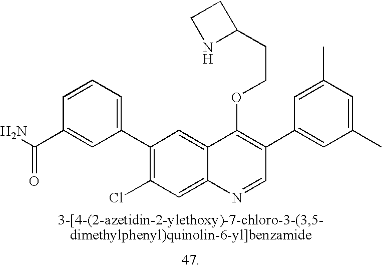 | 487.018 |
| 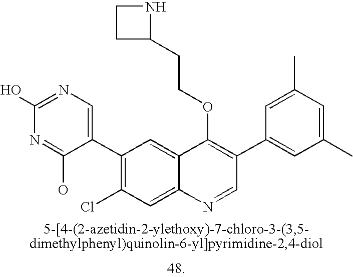 | 477.967 |
| 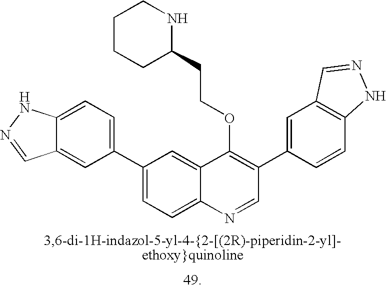 | 489.597 |
| 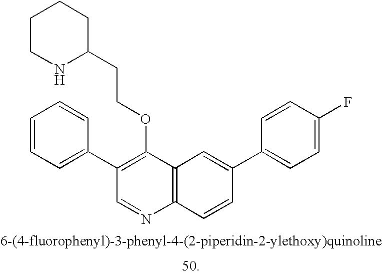 | 427.539 |
| 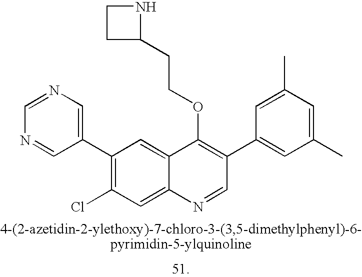 | 445.968 |
| 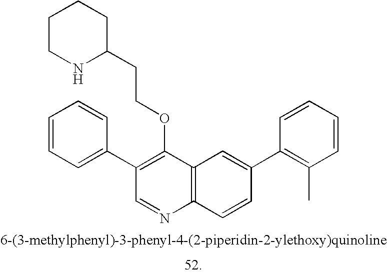 | 423.575 |
| 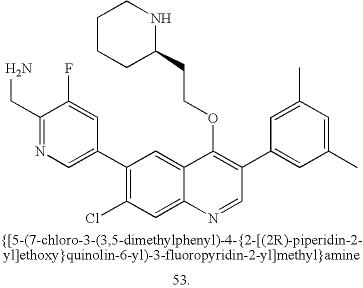 | 520.067 |
| 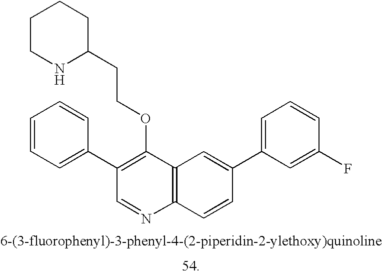 | 427.539 |
| 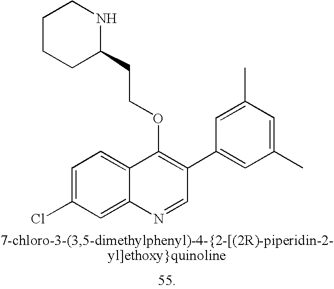 | 395.949 |
| 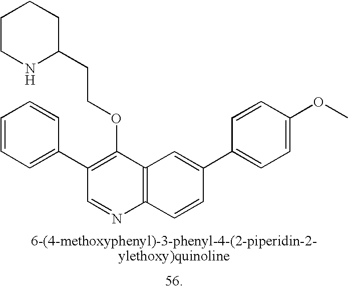 | 439.575 |
| 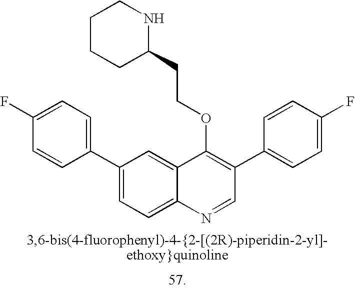 | 445.529 |
| 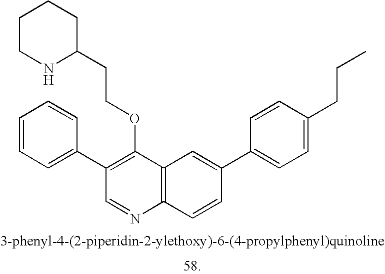 | 451.629 |
| 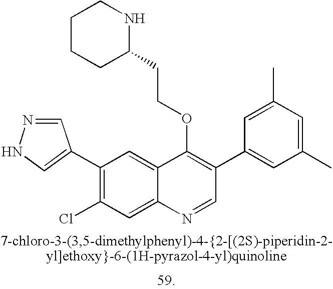 | 462.011 |
| 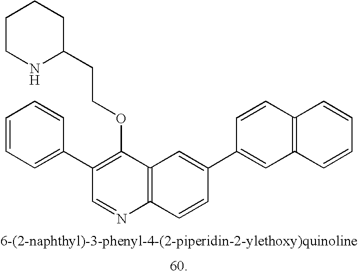 | 459.609 |
| 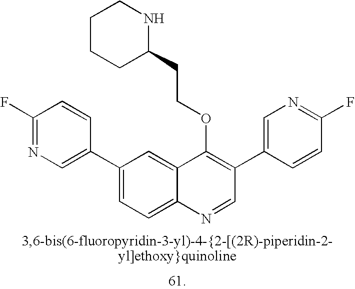 | 447.504 |
| 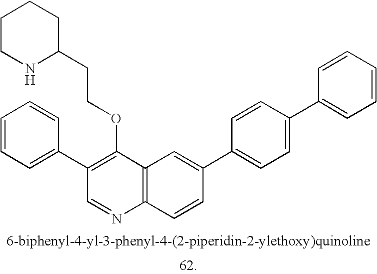 | 485.647 |
| 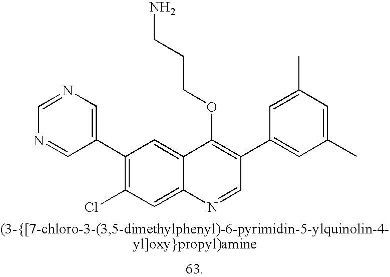 | 419.93 |
| 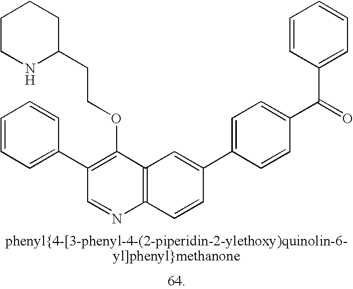 | 513.657 |
| 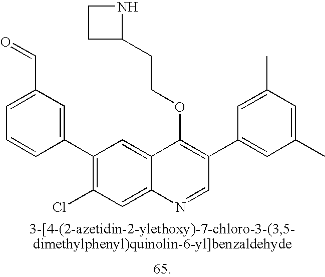 | 472.004 |
| 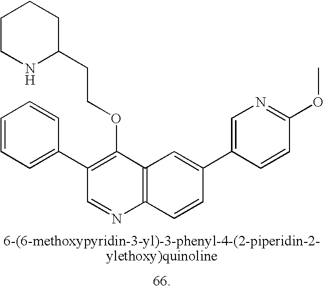 | 440.562 |
| 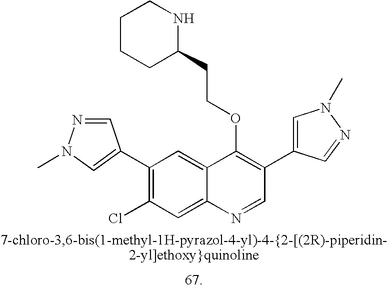 | 451.975 |
| 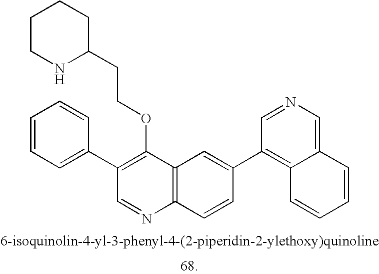 | 460.596 |
| 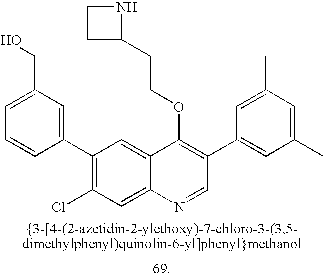 | 474.02 |
| 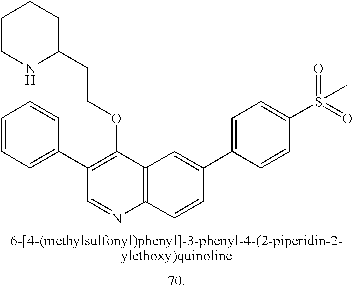 | 487.638 |
| 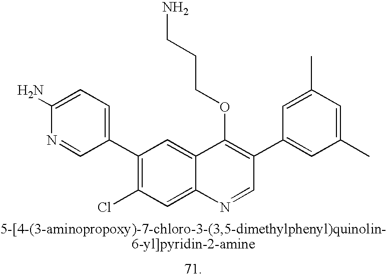 | 433.957 |
| 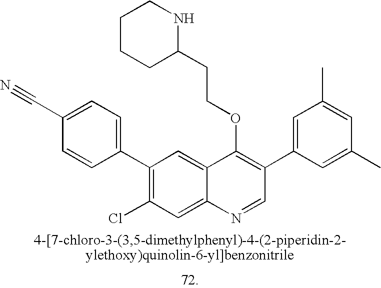 | 497.057 |
| 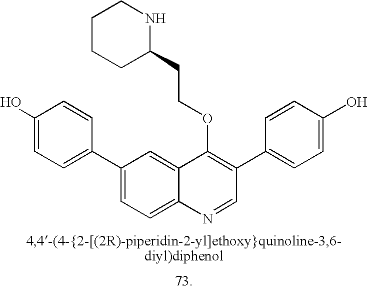 | 441.547 |
| 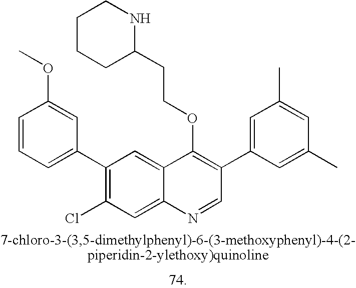 | 502.074 |
| 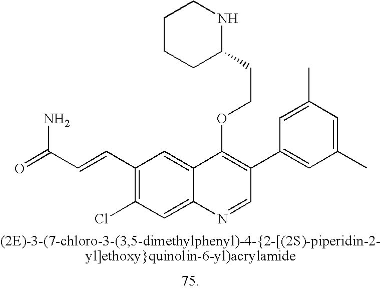 | 465.012 |
| 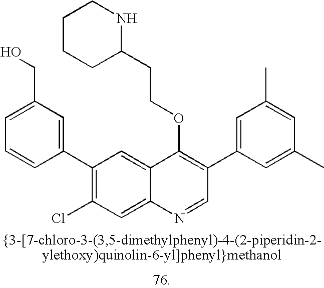 | 502.074 |
| 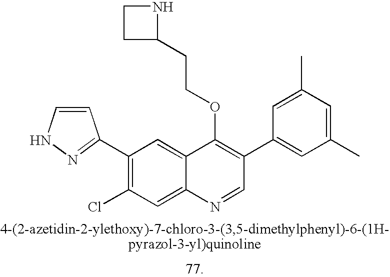 | 433.957 |
| 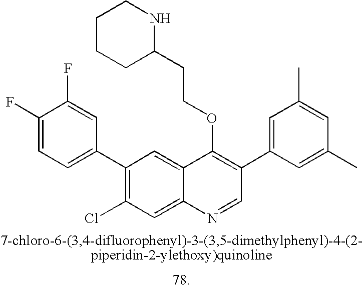 | 508.028 |
| 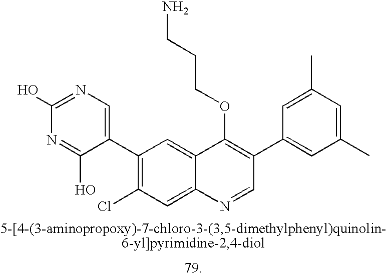 | 451.929 |
| 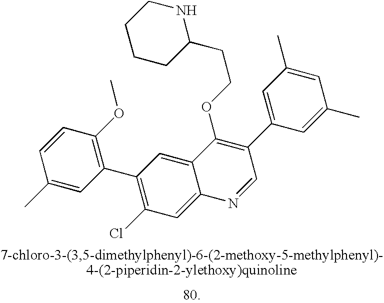 | 516.101 |
| 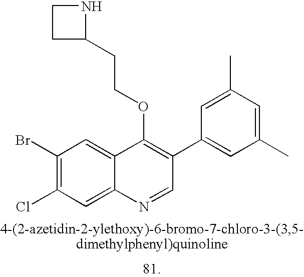 | 446.79 |
| 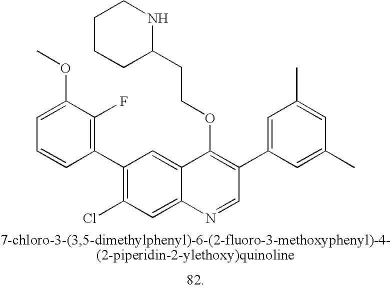 | 520.064 |
| 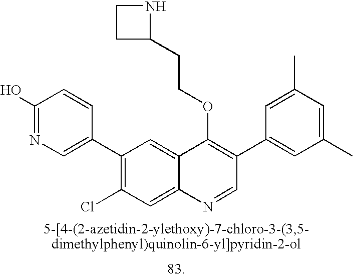 | 460.98 |
| 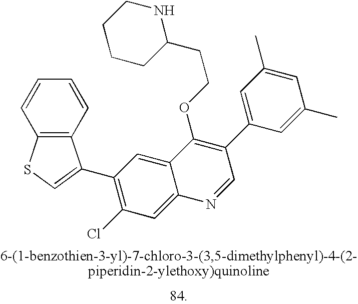 | 528.134 |
| 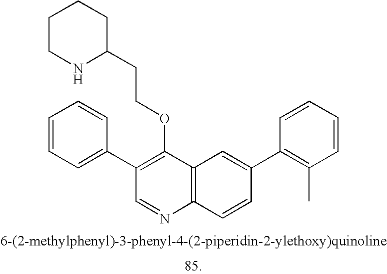 | 423.575 |
| 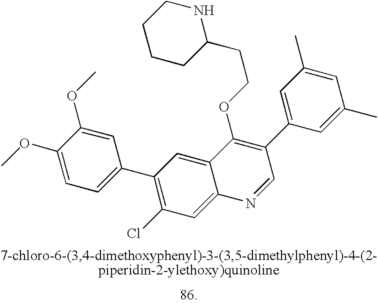 | 532.1 |
| 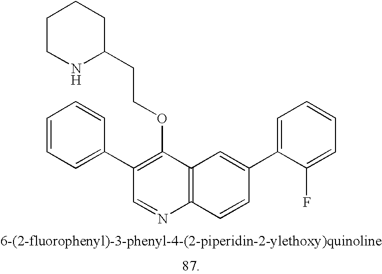 | 427.539 |
| 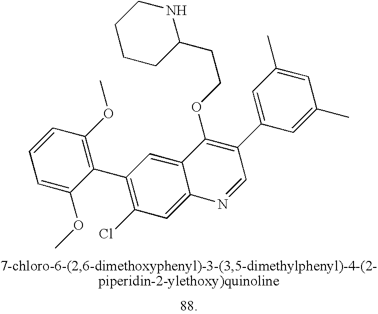 | 532.1 |
| 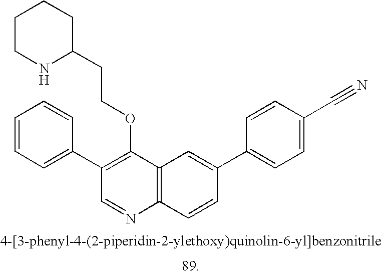 | 434.558 |
| 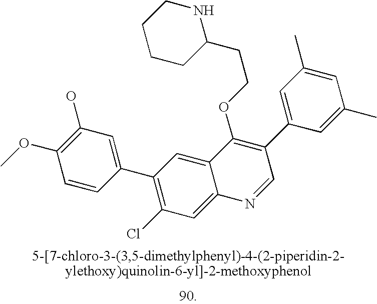 | 518.073 |
| 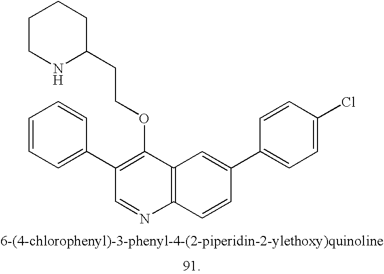 | 443.993 |
| 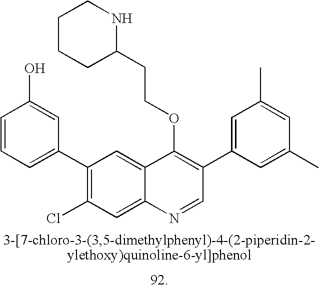 | 488.047 |
| 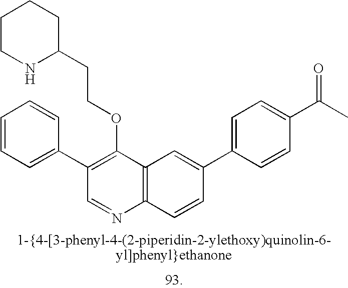 | 451.586 |
| 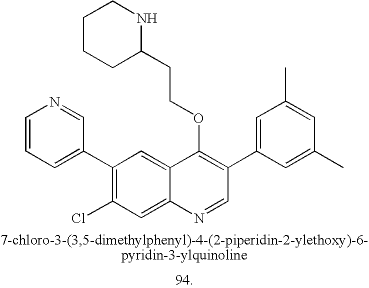 | 473.035 |
| 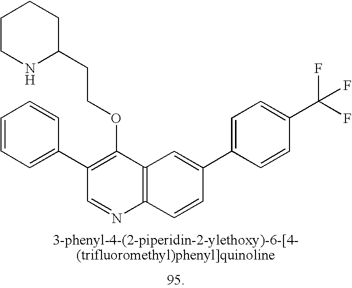 | 477.547 |
| 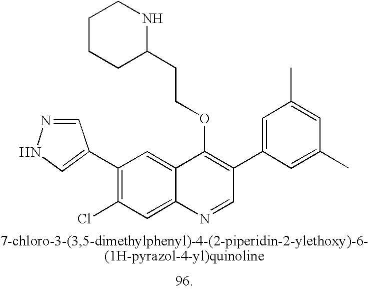 | 462.011 |
| 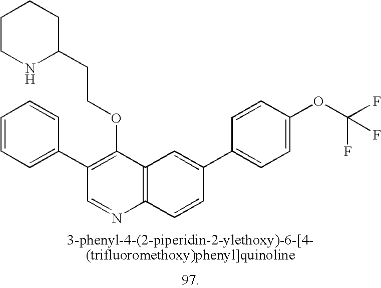 | 493.546 |
| 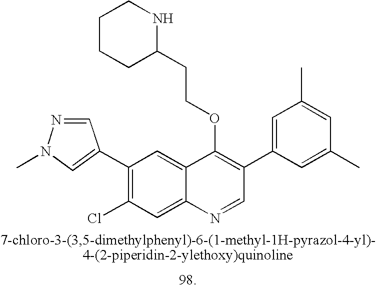 | 476.038 |
| 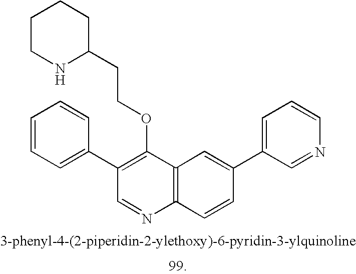 | 410.536 |
| 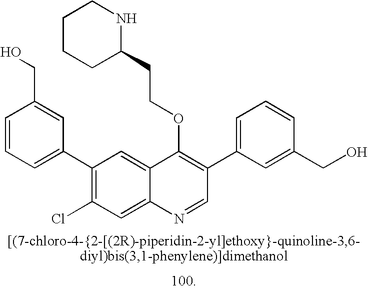 | 504.046 |
| 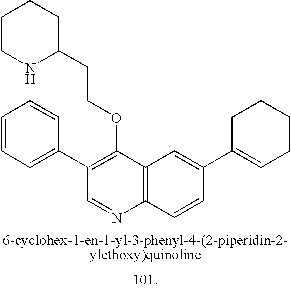 | 413.58 |
| 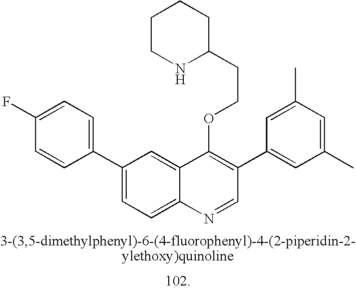 | 455.593 |
| 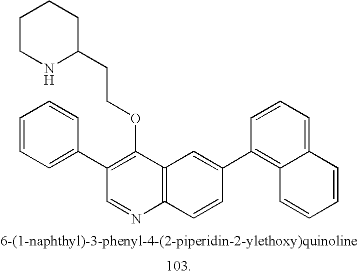 | 459.609 |
| 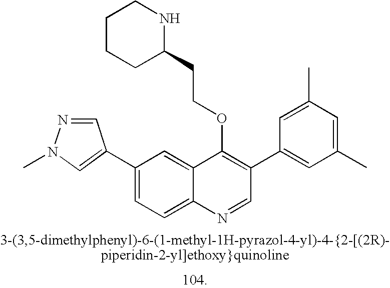 | 441.593 |
| 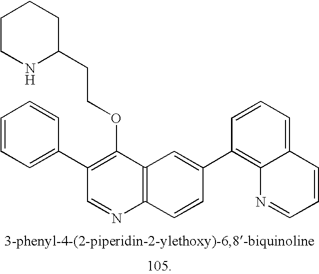 | 460.596 |
| 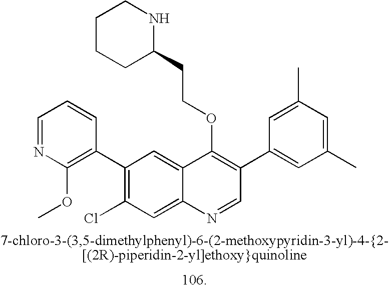 | 503.061 |
| 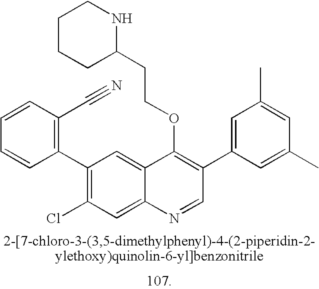 | 497.057 |
| 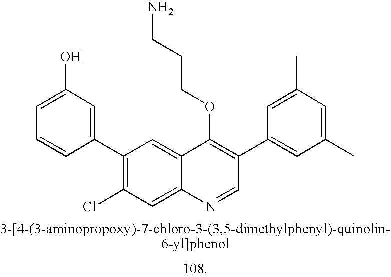 | 433.954 |
| 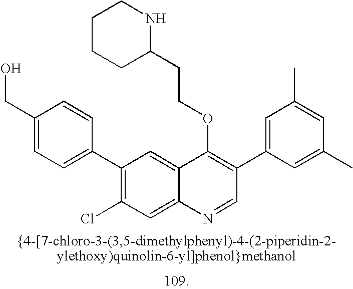 | 502.074 |
| 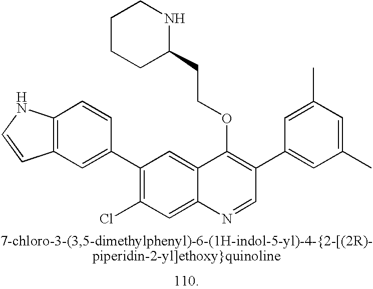 | 511.084 |
| 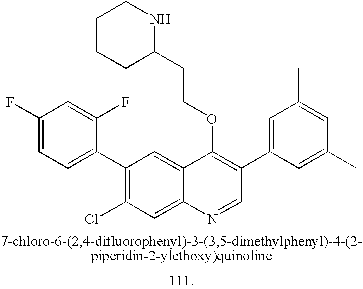 | 508.028 |
| 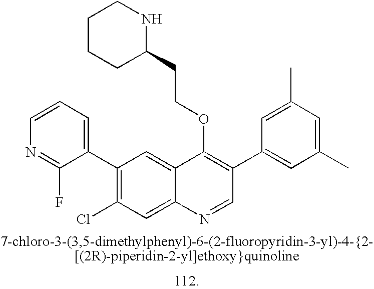 | 491.025 |
| 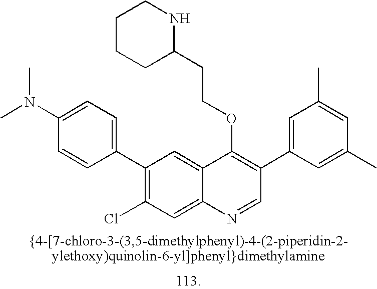 | 515.116 |
| 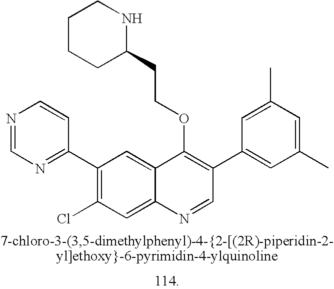 | 474.023 |
| 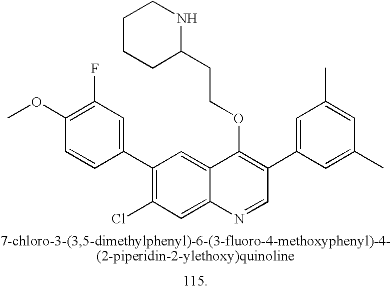 | 520.064 |
| 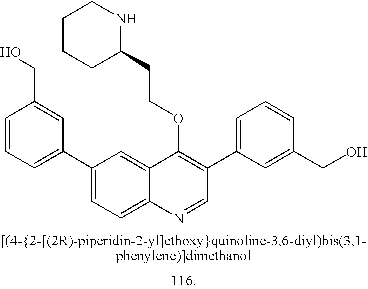 | 469.601 |
| 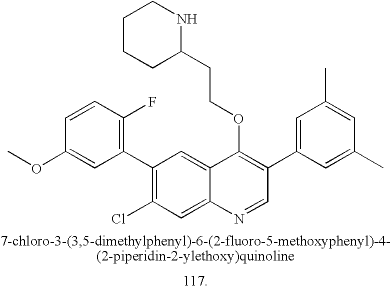 | 520.064 |
| 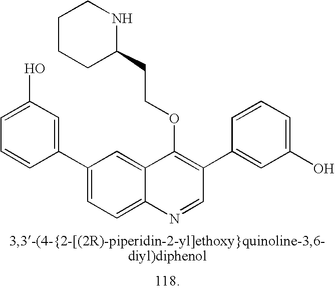 | 441.547 |
| 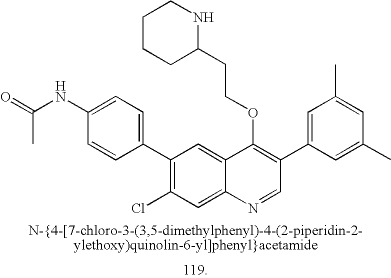 | 529.1 |
| 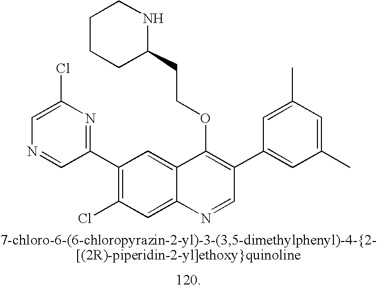 | 508.468 |
| 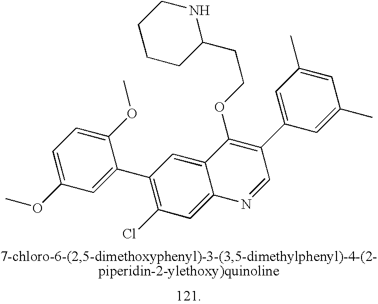 | 532.001 |
| 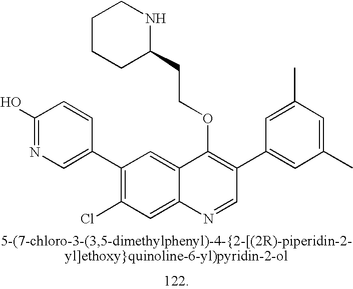 | 489.034 |
| 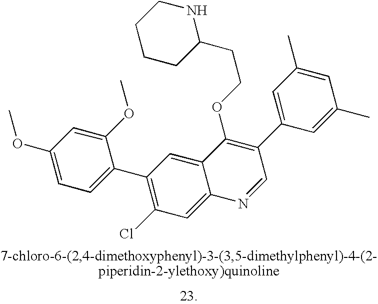 | 532.001 |
| 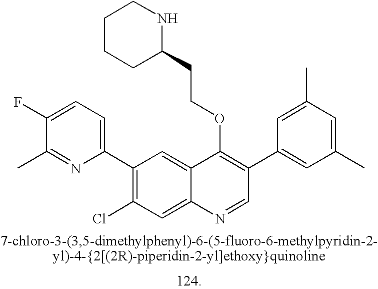 | 505.052 |
| 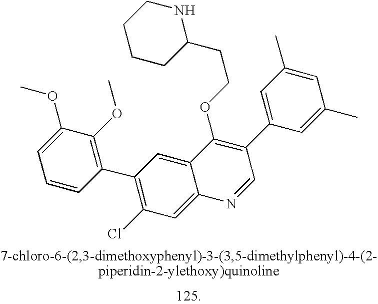 | 532.001 |
| 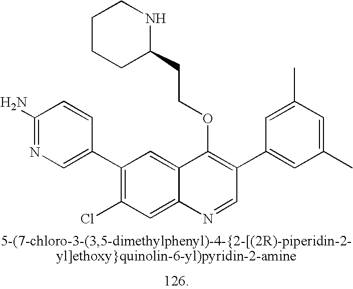 | 488.05 |
| 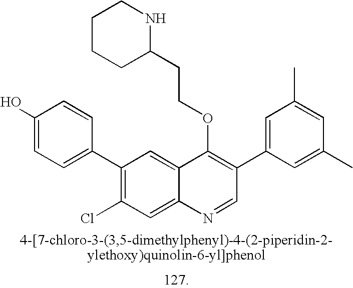 | 488.047 |
| 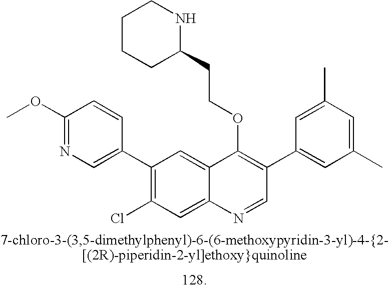 | 503.061 |
| 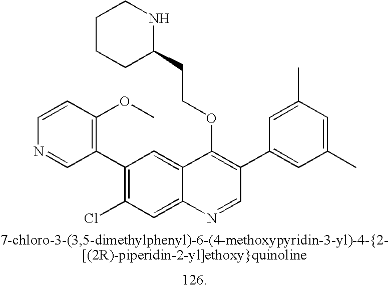 | 503.061 |
| 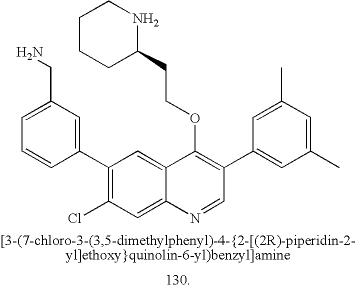 | 501.089 |
| 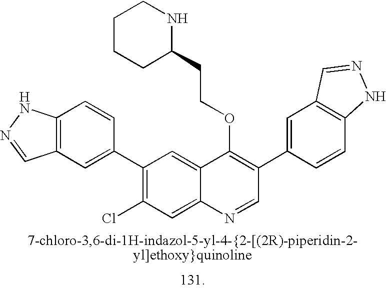 | 524.042 |
| 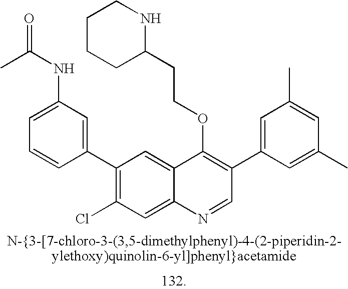 | 529.1 |
| 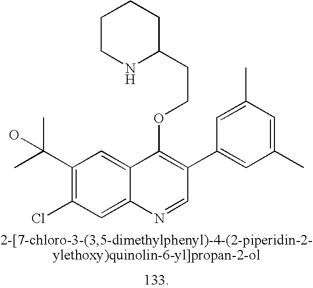 | 454.029 |

### SSTR Binding Assays

### General Overview:

Competitive binding studies are performed to assess the binding affinities of compounds of this invention for the cloned human and rodent somatostatin receptors. These studies rely on the ability of these compounds to compete with radiolabeled somatostatin for binding to the various somatostatin receptor subtypes. Competitive binding is performed by incubating serial dilutions of the compounds of interest with radiolabeled somatostatin and crude membrane fractions prepared from CHO cells stably expressing human or rodent somatostatin receptors. The amount of radiolabeled somatostatin bound to the membranes is then measured by scintography. By graphing the amount of bound radiolabeled somatostatin vs. the amount of test compound added to the binding reaction, the binding affinity of the test compounds can be calculated.

### Membrane Preparation:

Crude membrane fractions are prepared from Chinese hamster ovary (CHO) cells stably expressing one of the five human or rodent somatostatin receptor subtypes. The cells are grown to 85-100% confluence on standard tissue culture dishes in growth media containing alpha-minimal essential media (alpha-MEM, Gibco) with following additives: 10% fetal bovine serum (Gibco), 100 U/ml penicillin (Gibco), 100 ug/ml streptomycin (Gibco), 10 mM HEPES (Gibco), 0.5 mg/ml G-418 (Gibco). To prepare membranes, cells are washed once with 1× Dulbecco's phosphate buffered saline (Gibco) containing 10 mM HEPES (Gibco) then once with sodium-free binding buffer (50 mM Tris Base, 5 mM MgCl₂-6H₂0 and 1 mM EGTA adjusted to pH 7.8). The cells are then scraped into binding buffer containing a protease inhibitor cocktail (100 ug/ml pepstatin A (Sigma), 50 ug/ml leupeptin (Sigma), 25 ug/ml aprotinin (Sigma) and 10 mg/ml Bacitracin (USB Corporation)). The cells are centrifuged at 43,500×g, homogenized, and the resulting membranes are collected by centrifugation at 67,000×g. The membranes are then resuspended in binding buffer containing the protease inhibitor cocktail using a glass dounce homogenizer.

### Competitive Binding Assay:

The binding affinities of the compounds of the invention are measured using a competitive radioligand binding assay. The radiolabeled ligand (for example, 3-[¹²⁵I]iodotyrosyl¹¹ somatostatin-14(tyr11) from Amersham) and membrane fractions containing one of the SSTR subtypes are first mixed and incubated for 30 minutes at room temperature. Next, serial dilutions of the compounds of the invention dissolved in DMSO are added to the radioligand/membrane mixture and incubated at room temperature for 3 hours. Final assay conditions for the receptor binding assay are 0-10000 nM compound, 0.1 nM radiolabeled ¹²⁵I somatostatin 14 (Amersham), 2.5-50 ug membrane fraction, 0.5-2% DMSO brought up to a final assay volume of 1 ml in binding buffer+protease inhibitor cocktail. The membranes and bound radioligand are harvested by vacuum filtration onto Unifilter GF/B filter plates (Packard) pre-treated with 0.5% polyethyleneimine. Unbound radioligand is washed from the membranes with cold 50 mM Tris-HCl, pH 7.8. Microscint-20 scintillation fluid (Perkin Elmer) is added to the filter plates and the bound radioligand is counted on a scintillation counter. The Kᵢs are determined by plotting the bound radioligand counts vs. the amount of compound of the invention and using standard calculations (Harvey Motulsky and Richard Neubig, Current Protocols in Neuroscience, 1997, 7.5.1-7.5.55). The compounds of this invention have an IC₅₀ activity of <10 uM in the SSTR₂ binding assay.

### Functional Assay for SSTR₂ Agonists

### General Overview:

All five SSTR subtypes are Gᵢ coupled G-protein coupled receptors (GPCRS) that lead to decreases in intracellular cyclic AMP (cAMP) when activated by an agonist. Therefore, measurement of intracellular cAMP levels can be used to assess whether compounds of the invention are agonists of the SSTR subtypes (John Kelly, Troy Stevens, W. Joseph Thompson, and Roland Seifert, Current Protocols in Pharmacology, 2005, 2.2.1-2.2). One example of an intracellular cAMP assay is described below.

cAMP Assay Protocol:

One day prior to the assay, 40,000 Chinese hamster ovary (CHO) cells expressing the human somatostatin receptor subtype 2 are plated in each well of a 96-well tissue culture plate in growth media (alpha-minimal essential media (alpha-MEM, Gibco) with the following additives: 10% fetal bovine serum (Gibco), 100 U/ml penicillin (Gibco), 100 ug/ml streptomycin (Gibco), 10 mM HEPES (Gibco), 1.2 mM sodium hydroxide, 0.5 mg/ml G-418 (Gibco)). The cells are cultured overnight at 37° C., 5% CO₂ and 95% humidity. On the day of the assay, the media is aspirated and the cells are washed with 1× Dulbecco's phosphate buffered saline (Gibco). Next, 50 ul of assay buffer (1× Earle's Balanced Salt Solution (Gibco), 5 mM MgCl₂, 10 mM HEPES, 0.1% bovine serum albumin and 0.2 mM 3-Isobutyl-1-methylxanthine (IBMX, Biomol Research Labs)) is added and the cells are incubated for 15 minutes at room temperature. Various dilutions of the compounds of the invention are prepared in assay buffer and 50 ul of the dilutions are added to the cultured cells and incubated for 15 minutes at room temperature (the final concentration of the compounds of the invention are typically 0-10,000 nM). Next, 50 ul of assay buffer containing forskolin (Sigma) is added and incubated for 30 minutes at room temperature. The assay buffer, compound and forskolin are then aspirated and the cells are washed with 1× Dulbecco's phosphate buffered saline. The intracellular cAMP concentrations are then measured using a commercially available detection kit (for example, the cAMP SPA direct screening assay kit from Amersham). The measured intracellular cAMP concentrations are plotted vs. the concentration of the compounds of the invention and the EC₅₀ of the compounds are calculated using standard methods. The compounds of this invention have an IC50 activity of <10 uM in the SSTR₂ functional agonist assay.

What is claimed is:

1. A compound of Formula I: 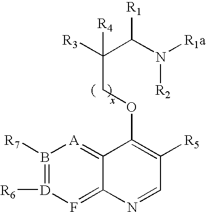
and pharmaceutically acceptable salts, esters, enantiomers, diastereomers or mixtures thereof wherein:
B and D independently represent carbon and nitrogen, A and F independently represent CH and nitrogen, provided that no more than 2 of A B, D and F are nitrogen at the same time;
R₁ and R₁a independently represent hydrogen, C₁-C₁₂ alkyl, (CH₂)ₘC₃-C₈ cycloalkyl; CF₃, CF₂H, CFH₂ or
R₁ and R₁a together with the nitrogen that R₁a is attached form a monocyclic or bicyclic heterocycle with 4-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR₁S(O)ₙalkyl,
R₂ represents hydrogen, C₁-C₁₂ alkyl, (CH₂)ₘC₃-C₈ cycloalkyl, COOR₁, said alkyl optionally substituted with 1 to 3 groups of halogen, C₁₋₆ alkyl, C₁₋₃ alkoxy, hydroxyl, CN, CF₃, (CH₂)ₘN(R₁)₂, (CH₂)ₘCOOR₁, C(O)N(R₁)₂, SO₂R₁, (CH₂)ₘS(O)ₙNR₁R₂, (C(NH)N(R₁)₂);
R₁ₐ and R₂ together with the nitrogen they are attached to form a monocyclic or bicyclic heterocycle with 4-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR₁, S(O)ₙalkyl;
R₃ and R₄ independently represent hydrogen, halogen, or C₁-C₁₂ alkyl; or
R₃ and R₄ together form a monocyclic or bicyclic carbocyclic or heterocyclic ring with 4-7 members in each ring and optionally containing one to three heteroatoms selected from N, O and S, said monocylcic or bicyclic carbocycle or heterocycle optionally substituted with one or more substituents selected from halogen, C₁₋₆ alkyl, C₁₋₃ alkoxy, (CH₂)ₘhydroxyl, CN, CF₃, (CH₂)ₘN(R₁)₂, (CH₂)ₘCOOR1, S(O)ₙalkyl; or
R₅ represents (CH₂)ₘC₆₋₁₀ aryl, (CH₂)ₘC₅₋₁₀ heterocyclyl, said aryl and heterocyclyl optionally substituted with 1 to 3 groups of halogen, C₁₋₆ alkyl, (CH₂)ₘC₃₋₇ cycloalkyl, CN, (CH₂)ₘOR₁, (CH₂)ₘCF₃, (CH₂)ₘCOOR₁, C(O)N(R₁)₂, (CH₂)ₘS(O)ₙR₁; (CH₂)ₘS(O)ₙNR₁R₂; (CH₂)ₘ[NR₁]S(O)ₙNR₁R₂; (CH₂)ₘ[NR₁]S(O)ₙR₁;
R₆ represents hydrogen, halogen, CN, C₁₋₆ alkyl, C₃₋₇ cycloalkyl, OR₁, CF₃, COOR₁, S(O)ₙR₁; S(O)₂NR₁ₐR₂; (CH₂)ₘC₅₋₁₀ heterocyclyl, —NS(O)₂NR₁ₐR₂, or is absent when D is nitrogen said alkyl and heterocyclyl optionally substituted with 1 to 3 groups of halogen, C₁₋₆ alkyl, (CH₂)ₘC₃₋₇ cycloalkyl, CN, (CH₂)ₘOR₁, CF₃, OCF₃, —NHC(O)R₁, CH(O), (CH₂)ₘC₆₋₁₀ aryl, C(O)C₆₋₁₀ aryl, (CH₂)ₘN(R₁)₂, C(O)N(R₁)₂, (CH₂)ₘCOOR₁, and (CH₂)ₘS(O)ₙR₁;
R₇ represents hydrogen, halogen, C₁₋₆ alkyl, C(O)OR₁, —C(CH₃)₂OH, —CH═CHC(O)N(R₁)₂, (CH₂)ₘC₃₋₇ cycloalkyl, CN, OR₁, CF₃, S(O)ₙR₁, CONR₉R¹⁰, NR₁CONR₁R₉, (CH₂)ₘC₆₋₁₀ aryl, (CH₂)ₘC₅₋₁₀ heterocyclyl, or is absent when B is nitrogen said alkyl, aryl and heterocyclyl optionally substituted with 1 to 3 groups of halogen, C₁₋₆ alkyl, (CH₂)ₘC₃₋₇ cycloalkyl, CN, (CH₂)ₘOR₁, CF₃, OCF₃, —NHC(O)R₁, CH(O), (CH₂)ₘC₆₋₁₀ aryl, C(O)C₆₋₁₀ aryl, (CH₂)ₘN(R₁)₂, C(O)N(R₁)₂, (CH₂)ₘCOOR₁, and (CH₂)ₘS(O)ₙR₁;
R₉ and R¹⁰ independently represent hydrogen, (CH₂)ₘ aryl, C₂-C₆ alkenyl, C₂-C₆ alkynyl, (CH₂)ₘ heterocyclyl, C₃-C₆ cycloalkyl, SO₂R⁷, and (C═O)N(R₁)₂, said alkyl, cycloalkyl, aryl, heterocylyl, alkenyl, and alkynyl optionally substituted with one or more substituents selected from halogen, C₁₋₆ alkyl, CN, CF₃, (CH₂)ₘN(R₁)₂, (CH₂)ₘOR₁, (CH₂)ₘCOOR₁, (CH₂)ₘS(O)ₙR₁;
R⁹ and R¹⁰ can be taken together with the nitrogen to which they are attached to form a monocyclic or bicyclic heterocycle with 5-7 members in each ring and optionally containing, in addition to the nitrogen, one or two additional heteroatoms selected from N, O and S, said monocylcic or bicyclic heterocycle optionally substituted with one or more substituents selected from halogen, C₁₋₆ alkyl, (CH₂)ₘOR₁, CN, CF₃, N(R₁)₂, COOR₁.
n is an integer from 0 to 2;
m is an integer from 0 to 6; and
x is an integer from 1 to 3.

2. A compound according to claim wherein A, B, D and F are all carbon, R₁ and R₁a together with the nitrogen that R₁a is attached form a monocyclic or bicyclic heterocycle, unsaturated or saturated, with 4-7 members in each ring and optionally containing in addition to the nitrogen, one or two additional heteroatoms selected from N, O and S, said monocylcic or bicyclic heterocycle optionally substituted with one or more substituents selected from halogen, C₁₋₆ alkyl, (CH₂)ₘOR₁, CN, CF₃, (CH₂)ₘN(R₁)₂, (CH₂)ₘCOOR₁, R₂ is hydrogen and R₃ and R₄ both are hydrogen.

3. A compound according to claim 2 wherein R₅ is aryl or heterocyclyl optionally substituted with one or more substituents selected from halogen, C₁₋₆ alkyl, (CH₂)ₘOR₁, CN, CF₃, (CH₂)ₘN(R₁)₂, (CH₂)ₘCOOR₁, and R₇ is aryl or heterocyclyl optionally substituted with one or more substituents selected from halogen, C₁₋₆ alkyl, (CH₂)ₘOR₁, CN, CF₃, (CH₂)ₘN(R₁)₂, (CH₂)ₘCOOR₁.

4. A compound according to claim 3 wherein R₅ is phenyl and R₇ is C₅₋₁₀ heteroaryl.

5. A compound according to claim 1 of structural formula II: 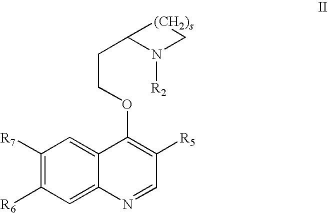
wherein, s is from 1 to 3 and R₂ is hydrogen, R₅, R₆ and R₇ are as described herein.

6. A compound according to claim 5 which is represented by structural formula IIa: 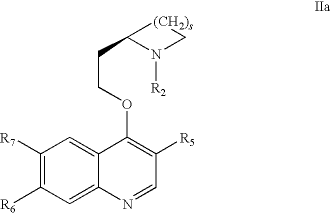
wherein, s is from 1 to 3 and R₂ is hydrogen, R₅, R₆ and R₇ are as described herein.

7. A compound according to claim 1 of structural formula III: 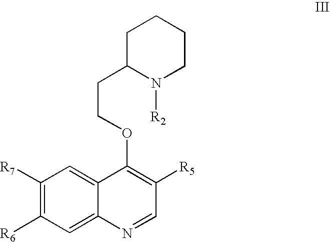
and pharmaceutically acceptable salts, esters, enantiomers, diastereomers or mixtures thereof wherein R₂ is hydrogen and R₅, R₆ and R₇ are as described herein.

8. A compound according to claim 1 which is selected from the group consisting of:
7-chloro-3-(3,5-dimethylphenyl)-6-(1-methyl-1H-pyrazol-4-yl)-4-{2-[(2R)-piperidin-2-yl]ethoxy}quinoline;
3-[4-(3-aminopropoxy)-7-chloro-3-(3,5-dimethylphenyl)quinolin-6-yl]benzamide;
3-(3,5-dimethylphenyl)-6-(1H-indazol-5-yl)-4-{2-[(2R)-piperidin-2-yl]ethoxy}quinoline;
2-[7-chloro-3-(3,5-dimethylphenyl)-4-(2-piperidin-2-ylethoxy)quinolin-6-yl]propan-2-ol;
7-chloro-3-(3,5-dimethylphenyl)-6-(1H-indazol-5-yl)-4-{2-[(2R)-piperidin-2-yl]ethoxy}quinoline;
7-chloro-3-(3,5-dimethylphenyl)-6-(1-methyl-1H-pyrazol-4-yl)-4-{2-[(2S)-piperidin-2-yl]ethoxy}-quinoline
3-(7-chloro-3-(3,5-dimethylphenyl)-4-{2-[(2R)-piperidin-2-yl]ethoxy}quinolin-6-yl)benzamide;
4-(7-chloro-3-(3,5-dimethylphenyl)-4-{2-[(2R)-piperidin-2-yl]ethoxy}quinolin-6-yl)benzamide;
3-(7-chloro-3-(3,5-dimethylphenyl)-4-{2-[(2R)-piperidin-2-yl]ethoxy}quinolin-6-yl)phenol;
7-chloro-3-(3,5-dimethylphenyl)-6-(2-methoxypyrimidin-5-yl)-4-{2-[(2R)-piperidin-2-yl]ethoxy}quinoline;
7-chloro-6-(2-chloropyridin-4-yl)-3-(3,5-dimethylphenyl)-4-{2-[(2R)-piperidin-2-yl]ethoxy}quinoline;
{3-[4-(3-aminopropoxy)-7-chloro-3-(3,5-dimethylphenyl)quinolin-6-yl]phenyl}methanol;
7-chloro-3-(3,5-dimethylphenyl)-6-(4-fluorophenyl)-4-(2-piperidin-2-ylethoxy)quinoline;
7-chloro-3-(3,5-dimethylphenyl)-4-(2-piperidin-2-ylethoxy)-6-(1,3-thiazol-2-yl)quinoline
7-chloro-3-(3,5-dimethylphenyl)-4-{2-[(2R)-piperidin-2-yl]ethoxy}-6-(1H-pyrazol-5-yl)quinoline;
(3-{[7-chloro-3-(3,5-dimethylphenyl)-6-(1-methyl-1H-pyrazol-3-yl)quinolin-4-yl]oxy}propyl)amine;
7-chloro-3-(3,5-dimethylphenyl)-6-(2-fluoropyridin-4-yl)-4-{2-[(2R)-piperidin-2-yl]ethoxy}quinoline;
6-bromo-7-chloro-3-(3,5-dimethylphenyl)-4-{2-[(2R)-piperidin-2-yl]ethoxy}quinoline;
4-(7-chloro-3-(3,5-dimethylphenyl)-4-{2-[(2R)-piperidin-2-yl]ethoxy}quinolin-6-yl)phenol;
4,4′-(7-chloro-4-{2-[(2R)-piperidin-2-yl]ethoxy}quinoline-3,6-diyl)diphenol;
[3-(7-chloro-3-(3,5-dimethylphenyl)-4-{2-[(2R)-piperidin-2-yl]ethoxy}quinolin-6-yl)phenyl]methanol;
5-(7-chloro-3-(3,5-dimethylphenyl)-4-{2-[(2R)-piperidin-2-yl]ethoxy}quinolin-6-yl)pyrimidine-2,4-diol;
[4-(7-chloro-3-(3,5-dimethylphenyl)-4-{2-[(2R)-piperidin-2-yl]ethoxy}quinolin-6-yl)phenyl]methanol;
methyl 7-chloro-3-(3,5-dimethylphenyl)-4-(2-piperidin-2-ylethoxy)quinoline-6-carboxylate;
7-chloro-3-(3,5-dimethylphenyl)-6-(1H-indazol-6-yl)-4-(2-piperidin-2-ylethoxy)quinoline;
3,3′-(7-chloro-4-{2-[(2R)-piperidin-2-yl]ethoxy}quinoline-3,6-diyl)dibenzamide;
7-chloro-3-(3,5-dimethylphenyl)-4-{2-[(2R)-piperidin-2-yl]ethoxy}-6-pyridin-4-ylquinoline;
3-(3,5-dimethylphenyl)-4-(2-piperidin-2-ylethoxy)-6,7-di-1,3-thiazol-2-ylquinoline;
7-chloro-3-(3,5-dimethylphenyl)-6-(6-fluoropyridin-3-yl)-4-{2-[(2R)-piperidin-2-yl]ethoxy}quinoline;
7-chloro-3,6-bis(6-fluoropyridin-3-yl)-4-{2-[(2R)-piperidin-2-yl]ethoxy}quinoline;
7-chloro-6-(6-chloropyridin-3-yl)-3-(3,5-dimethylphenyl)-4-{2-[(2R)-piperidin-2-yl]ethoxy}quinoline;
4-(2-azetidin-2-ylethoxy)-7-chloro-3-(3,5-dimethylphenyl)-6-(1H-indazol-5-yl)quinoline;
7-chloro-3-(3,5-dimethylphenyl)-4-{2-[(2S)-piperidin-2-yl]ethoxy}-6-pyridin-3-ylquinoline;
4-(2-azetidin-2-ylethoxy)-7-chloro-3-(3,5-dimethylphenyl)-6-(1-methyl-1H-pyrazol-3-yl)quinoline;
7-chloro-3-(3,5-dimethylphenyl)-4-{2-[(2R)-piperidin-2-yl]ethoxy}-6-(1H-pyrazol-4-yl)quinoline;
3-(7-chloro-3-(3,5-dimethylphenyl)-4-{2-[(2R)-piperidin-2-yl]ethoxy}quinolin-6-yl)-N-methylbenzamide;
7-chloro-3-(3,5-dimethylphenyl)-6-(5-methoxypyridin-3-yl)-4-{2-[(2R)-piperidin-2-yl]ethoxy}quinoline;
3,3′-(4-{2-[(2R)-piperidin-2-yl]ethoxy}quinoline-3,6-diyl)dibenzamide;
3,3′-(7-chloro-4-{2-[(2R)-piperidin-2-yl]ethoxy}quinoline-3,6-diyl)diphenol;
4-(2-azetidin-2-ylethoxy)-7-chloro-3-(3,5-dimethylphenyl)-6-(6-fluoropyridin-3-yl)quinoline;
7-chloro-3,6-bis(4-fluorophenyl)-4-{2-[(2R)-piperidin-2-yl]ethoxy}quinoline;
4-(2-azetidin-2-ylethoxy)-7-chloro-3-(3,5-dimethylphenyl)-6-pyridin-4-ylquinoline;
(2E)-3-(7-chloro-3-(3,5-dimethylphenyl)-4-{2-[(2R)-piperidin-2-yl]ethoxy}quinolin-6-yl)acrylamide;
5-[4-(2-azetidin-2-ylethoxy)-7-chloro-3-(3,5-dimethylphenyl)quinolin-6-yl]pyridin-2-amine;
(3-{[7-chloro-3-(3,5-dimethylphenyl)-6-(1H-indazol-5-yl)quinolin-4-yl]oxy}propyl)amine;
4-(2-azetidin-2-ylethoxy)-7-chloro-3-(3,5-dimethylphenyl)-6-pyridin-3-ylquinoline;
3-[4-(2-azetidin-2-ylethoxy)-7-chloro-3-(3,5-dimethylphenyl)quinolin-6-yl]phenol;
3,6-bis(1-methyl-1H-pyrazol-4-yl)-4-{2-[(2S)-piperidin-2-yl]ethoxy}quinoline;
3-[4-(2-azetidin-2-ylethoxy)-7-chloro-3-(3,5-dimethylphenyl)quinolin-6-yl]benzamide;
5-[4-(2-azetidin-2-ylethoxy)-7-chloro-3-(3,5-dimethylphenyl)quinolin-6-yl]pyrimidine-2,4-diol;
3,6-di-1H-indazol-5-yl-4-{2-[(2R)-piperidin-2-yl]ethoxy}quinoline;
6-(4-fluorophenyl)-3-phenyl-4-(2-piperidin-2-ylethoxy)quinoline;
4-(2-azetidin-2-ylethoxy)-7-chloro-3-(3,5-dimethylphenyl)-6-pyrimidin-5-ylquinoline;
6-(3-methylphenyl)-3-phenyl-4-(2-piperidin-2-ylethoxy)quinoline;
{[5-(7-chloro-3-(3,5-dimethylphenyl)-4-{2-[(2R)-piperidin-2-yl]ethoxy}quinolin-6-yl)-3-fluoropyridin-2-yl]methyl}amine;
6-(3-fluorophenyl)-3-phenyl-4-(2-piperidin-2-ylethoxy)quinoline;
7-chloro-3-(3,5-dimethylphenyl)-4-{2-[(2R)-piperidin-2-yl]ethoxy}quinoline;
6-(4-methoxyphenyl)-3-phenyl-4-(2-piperidin-2-ylethoxy)quinoline;
3,6-bis(4-fluorophenyl)-4-{2-[(2R)-piperidin-2-yl]ethoxy}quinoline;
3-phenyl-4-(2-piperidin-2-ylethoxy)-6-(4-propylphenyl)quinoline;
7-chloro-3-(3,5-dimethylphenyl)-4-{2-[(2S)-piperidin-2-yl]ethoxy}-6-(1H-pyrazol-4-yl)quinoline;
6-(2-naphthyl)-3-phenyl-4-(2-piperidin-2-ylethoxy)quinoline;
3,6-bis(6-fluoropyridin-3-yl)-4-{2-[(2R)-piperidin-2-yl]ethoxy}quinoline;
6-biphenyl-4-yl-3-phenyl-4-(2-piperidin-2-ylethoxy)quinoline;
(3-{[7-chloro-3-(3,5-dimethylphenyl)-6-pyrimidin-5-ylquinolin-4-yl]oxy}propyl)amine;
phenyl {4-[3-phenyl-4-(2-piperidin-2-ylethoxy)quinolin-6-yl]phenyl}methanone;
3-[4-(2-azetidin-2-ylethoxy)-7-chloro-3-(3,5-dimethylphenyl)quinolin-6-yl]benzaldehyde;
6-(6-methoxypyridin-3-yl)-3-phenyl-4-(2-piperidin-2-ylethoxy)quinoline;
7-chloro-3,6-bis(1-methyl-1H-pyrazol-4-yl)-4-{2-[(2R)-piperidin-2-yl]ethoxy}quinoline;
6-isoquinolin-4-yl-3-phenyl-4-(2-piperidin-2-ylethoxy)quinoline;
{3-[4-(2-azetidin-2-ylethoxy)-7-chloro-3-(3,5-dimethylphenyl)quinolin-6-yl]phenyl}methanol;
6-[4-(methylsulfonyl)phenyl]-3-phenyl-4-(2-piperidin-2-ylethoxy)quinoline;
5-[4-(3-aminopropoxy)-7-chloro-3-(3,5-dimethylphenyl)quinolin-6-yl]pyridin-2-amine;
4-[7-chloro-3-(3,5-dimethylphenyl)-4-(2-piperidin-2-ylethoxy)quinolin-6-yl]benzonitrile;
4,4′-(4-{2-[(2R)-piperidin-2-yl]ethoxy}quinoline-3,6-diyl)diphenol;
7-chloro-3-(3,5-dimethylphenyl)-6-(3-methoxyphenyl)-4-(2-piperidin-2-ylethoxy)quinoline;
(2E)-3-(7-chloro-3-(3,5-dimethylphenyl)-4-{2-[(2S)-piperidin-2-yl]ethoxy}quinolin-6-yl)acrylamide;
{3-[7-chloro-3-(3,5-dimethylphenyl)-4-(2-piperidin-2-ylethoxy)quinolin-6-yl]phenyl}methanol;
4-(2-azetidin-2-ylethoxy)-7-chloro-3-(3,5-dimethylphenyl)-6-(1H-pyrazol-3-yl)quinoline;
7-chloro-6-(3,4-difluorophenyl)-3-(3,5-dimethylphenyl)-4-(2-piperidin-2-ylethoxy)quinoline;
5-[4-(3-aminopropoxy)-7-chloro-3-(3,5-dimethylphenyl)quinolin-6-yl]pyrimidine-2,4-diol;
7-chloro-3-(3,5-dimethylphenyl)-6-(2-methoxy-5-methylphenyl)-4-(2-piperidin-2-ylethoxy)quinoline;
4-(2-azetidin-2-ylethoxy)-6-bromo-7-chloro-3-(3,5-dimethylphenyl)quinoline;
7-chloro-3-(3,5-dimethylphenyl)-6-(2-fluoro-3-methoxyphenyl)-4-(2-piperidin-2-ylethoxy)quinoline;
5-[4-(2-azetidin-2-ylethoxy)-7-chloro-3-(3,5-dimethylphenyl)quinolin-6-yl]pyridin-2-ol;
6-(1-benzothien-3-yl)-7-chloro-3-(3,5-dimethylphenyl)-4-(2-piperidin-2-ylethoxy)quinoline;
7-chloro-3-(3,5-dimethylphenyl)-4-piperidin-1-ylquinoline;
N-{3-[7-chloro-3-(3,5-dimethylphenyl)-4-(2-piperidin-2-ylethoxy)quinolin-6-yl]phenyl}acetamide;
6-(2-methylphenyl)-3-phenyl-4-(2-piperidin-2-ylethoxy)quinoline;
7-chloro-6-(3,4-dimethoxyphenyl)-3-(3,5-dimethylphenyl)-4-(2-piperidin-2-ylethoxy)quinoline;
6-(2-fluorophenyl)-3-phenyl-4-(2-piperidin-2-ylethoxy)quinoline;
7-chloro-6-(2,6-dimethoxyphenyl)-3-(3,5-dimethylphenyl)-4-(2-piperidin-2-ylethoxy)quinoline;
4-[3-phenyl-4-(2-piperidin-2-ylethoxy)quinolin-6-yl]benzonitrile;
5-[7-chloro-3-(3,5-dimethylphenyl)-4-(2-piperidin-2-ylethoxy)quinolin-6-yl]-2-methoxyphenol;
6-(4-chlorophenyl)-3-phenyl-4-(2-piperidin-2-ylethoxy)quinoline;
3-[7-chloro-3-(3,5-dimethylphenyl)-4-(2-piperidin-2-ylethoxy)quinolin-6-yl]phenol;
1-{4-[3-phenyl-4-(2-piperidin-2-ylethoxy)quinolin-6-yl]phenyl}ethanone;
7-chloro-3-(3,5-dimethylphenyl)-4-(2-piperidin-2-ylethoxy)-6-pyridin-3-ylquinoline;
3-phenyl-4-(2-piperidin-2-ylethoxy)-6-[4-(trifluoromethyl)phenyl]quinoline;
7-chloro-3-(3,5-dimethylphenyl)-4-(2-piperidin-2-ylethoxy)-6-(1H-pyrazol-4-yl)quinoline;
3-phenyl-4-(2-piperidin-2-ylethoxy)-6-[4-(trifluoromethoxy)phenyl]quinoline;
7-chloro-3-(3,5-dimethylphenyl)-6-(1-methyl-1H-pyrazol-4-yl)-4-(2-piperidin-2-ylethoxy)quinoline;
3-phenyl-4-(2-piperidin-2-ylethoxy)-6-pyridin-3-ylquinoline;
[(7-chloro-4-{2-[(2R)-piperidin-2-yl]ethoxy}quinoline-3,6-diyl)bis(3,1-phenylene)]dimethanol;
6-cyclohex-1-en-1-yl-3-phenyl-4-(2-piperidin-2-ylethoxy)quinoline;
3-(3,5-dimethylphenyl)-6-(4-fluorophenyl)-4-(2-piperidin-2-ylethoxy)quinoline;
6-(1-naphthyl)-3-phenyl-4-(2-piperidin-2-ylethoxy)quinoline;
3-(3,5-dimethylphenyl)-6-(1-methyl-1H-pyrazol-4-yl)-4-{2-[(2R)-piperidin-2-yl]ethoxy}quinoline;
3-phenyl-4-(2-piperidin-2-ylethoxy)-6,8′-biquinoline;
7-chloro-3-(3,5-dimethylphenyl)-6-(2-methoxypyridin-3-yl)-4-{2-[(2R)-piperidin-2-yl]ethoxy}quinoline;
2-[7-chloro-3-(3,5-dimethylphenyl)-4-(2-piperidin-2-ylethoxy)quinolin-6-yl]benzonitrile;
3-[4-(3-aminopropoxy)-7-chloro-3-(3,5-dimethylphenyl)quinolin-6-yl]phenol;
{4-[7-chloro-3-(3,5-dimethylphenyl)-4-(2-piperidin-2-ylethoxy)quinolin-6-yl]phenyl}methanol;
7-chloro-3-(3,5-dimethylphenyl)-6-(1H-indol-5-yl)-4-{2-[(2R)-piperidin-2-yl]ethoxy}quinoline;
7-chloro-6-(2,4-difluorophenyl)-3-(3,5-dimethylphenyl)-4-(2-piperidin-2-ylethoxy)quinoline;
7-chloro-3-(3,5-dimethylphenyl)-6-(2-fluoropyridin-3-yl)-4-{2-[(2R)-piperidin-2-yl]ethoxy}quinoline;
{4-[7-chloro-3-(3,5-dimethylphenyl)-4-(2-piperidin-2-ylethoxy)quinolin-6-yl]phenyl}dimethylamine;
7-chloro-3-(3,5-dimethylphenyl)-4-{2-[(2R)-piperidin-2-yl]ethoxy}-6-pyrimidin-4-ylquinoline;
7-chloro-3-(3,5-dimethylphenyl)-6-(3-fluoro-4-methoxyphenyl)-4-(2-piperidin-2-ylethoxy)quinoline;
[(4-{2-[(2R)-piperidin-2-yl]ethoxy}quinoline-3,6-diyl)bis(3,1-phenylene)]dimethanol;
7-chloro-3-(3,5-dimethylphenyl)-6-(2-fluoro-5-methoxyphenyl)-4-(2-piperidin-2-ylethoxy)quinoline;
3,3′-(4-{2-[(2R)-piperidin-2-yl]ethoxy}quinoline-3,6-diyl)diphenol;
N-{4-[7-chloro-3-(3,5-dimethylphenyl)-4-(2-piperidin-2-ylethoxy)quinolin-6-yl]phenyl}acetamide;
7-chloro-6-(6-chloropyrazin-2-yl)-3-(3,5-dimethylphenyl)-4-{2-[(2R)-piperidin-2-yl]ethoxy}quinoline;
7-chloro-6-(2,5-dimethoxyphenyl)-3-(3,5-dimethylphenyl)-4-(2-piperidin-2-ylethoxy)quinoline;
5-(7-chloro-3-(3,5-dimethylphenyl)-4-{2-[(2R)-piperidin-2-yl]ethoxy}quinolin-6-yl)pyridin-2-ol;
7-chloro-6-(2,4-dimethoxyphenyl)-3-(3,5-dimethylphenyl)-4-(2-piperidin-2-ylethoxy)quinoline;
7-chloro-3-(3,5-dimethylphenyl)-6-(5-fluoro-6-methylpyridin-2-yl)-4-{2-[(2R)-piperidin-2-yl]ethoxy}quinoline;
7-chloro-6-(2,3-dimethoxyphenyl)-3-(3,5-dimethylphenyl)-4-(2-piperidin-2-ylethoxy)quinoline;
5-(7-chloro-3-(3,5-dimethylphenyl)-4-{2-[(2R)-piperidin-2-yl]ethoxy}quinolin-6-yl)pyridin-2-amine;
4-[7-chloro-3-(3,5-dimethylphenyl)-4-(2-piperidin-2-ylethoxy)quinolin-6-yl]phenol;
7-chloro-3-(3,5-dimethylphenyl)-6-(6-methoxypyridin-3-yl)-4-{2-[(2R)-piperidin-2-yl]ethoxy}quinoline;
7-chloro-3-(3,5-dimethylphenyl)-6-(4-methoxypyridin-3-yl)-4-{2-[(2R)-piperidin-2-yl]ethoxy}quinoline;
[3-(7-chloro-3-(3,5-dimethylphenyl)-4-{2-[(2R)-piperidin-2-yl]ethoxy}quinolin-6-yl)benzyl]amine;
7-chloro-3,6-di-1H-indazol-5-yl-4-{2-[(2R)-piperidin-2-yl]ethoxy}quinoline or
their pharmaceutically acceptable salts, esters, enantiomers, diastereomers or mixtures thereof.

9. A composition according to claim 1, comprising a compound of formula I and a pharmaceutically acceptable carrier.

10. Use of the compound of claim 1 for the manufacture of a medicament in the treatment and prevention of diabetes, diabetic retinopathy, neuropathy and nephropathy.
